(19) United States
(12) Patent Application Publication
(10) Pub. No.: US 2026/0076900 A1
(43) Pub. Date: Mar. 19, 2026

(54) IPRATROPIUM DELIVERY COMPOSITIONS, DEVICES AND METHODS

(71) Applicant: Solstice Advanced Materials US, Inc., Morris Plains, NJ (US)

(72) Inventors: Erik Boldt, Lancaster, NY (US); Gregory Laurence Smith, Niagara Falls (CA)

(73) Assignee: Solstice Advanced Materials US, Inc., Morris Plains, NJ (US)

(21) Appl. No.: 19/328,020

(22) Filed: Sep. 12, 2025

Related U.S. Application Data
(60) Provisional application No. 63/695,332, filed on Sep. 16, 2024.

(51) Int. Cl. A61K 9/00; A61K 31/439; A61K 47/12

(52) U.S. Cl. CPC A61K 9/008; A61K 31/439; A61K 47/12

(57) ABSTRACT

Disclosed are medicinal compositions, and devices, methods and systems which provide ipratropium bromide carried by at least 1234ze(E) and ethanol.

### CROSS REFERENCE TO RELATED APPLICATIONS

This invention is related to and claims the priority benefit of U.S. Provisional Application No. 63/695,332, filed Sep. 16, 2024, which is incorporated herein by reference.

### FIELD OF THE INVENTION

This invention relates to medicament delivery compositions, systems, devices and methods. In particular aspects, this invention relates to medicinal aerosol compositions, methods and devices used for metered dose delivery of ipratropium.

### BACKGROUND OF THE INVENTION

Pressurized metered dose inhalers (pMDIs) have long been used to deliver medicaments, including anticholinergics and bronchodilators, to the areas of patients needing treatment for various conditions. Ipratropium is used as an anticholinergic and/or a bronchodilators, and has been used for the treatment of various maladies, including chronic obstructive pulmonary disease (COPD), Parkinson's disease and urinary incontinence.

pMDIs may be used to deliver medicaments in a solubilized form or as a suspension. Typically, pMDIs use a relatively high vapor pressure propellant to carry and expel aerosolized droplets containing the API away from the pMDI and towards the respiratory tract when the pMDI is activated. The propellant/carrier for the active pharmaceutical ingredient (sometimes referred to herein as “API”) must be safe for the patients' use and be pharmaceutically acceptable. For certain APIs, including ipratropium, the API is preferably in the pMDI as a solute in a solvent carrier. The carrier can comprise one or more materials that act as a propellant for the pMDI and as a solvent for the API.

Carrier properties can have an impact on pMDI performance. For example, the one or more materials that make up the carrier should preferably have an appropriate boiling point and vapor pressure so that it can be liquefied in a closed container at room temperature but develop a high enough pressure when the pMDI is activated to deliver the drug as an atomized composition even at low ambient temperatures. Further, the carrier should be of low acute and chronic toxicity. It should have a high degree of chemical stability with the API in solution therein and with the container and the metallic and non-metallic components of the pMDI device. The carrier should also have a low propensity to extract low molecular weight substances from any elastomeric materials in the pMDI device. Finally, the carrier should not present a significant flammability risk to the patient in use. In particular, it should form a non-flammable or low flammability mixture when mixed with air in the respiratory tract. Environmentally desirable properties, such as low GWP and low ODP, are also generally highly desirable.

As will be appreciated by those of skill in the art, the efficacy of a pMDI delivery system depends, in part, on the size distribution of the aerosol droplets/particles that are introduced, typically via the mouth of the user. Generally speaking, the aerosol which is produced by the activation of a pMDI will have a distribution of droplets that immediately or essentially immediately result in aerosolized particles as a result of the evaporation of the carrier components. The particle sizes produced can range from extremely small to relatively large, and it is generally highly desirable for the pMDI to produce a size distribution tending toward the small particles (e.g., in the range of less from about 1 micron to about 5 micron). This desire exists for two reasons. First, the goal of ipratropium pMDIs designed for introduction via the mouth and/or nose is to deliver the medication to the lungs of the user for absorption into the body, and smaller particle sizes in this range are most readily absorbed into the lungs. Second, it is known that larger particle sizes (e.g., greater than 5 micron) tend to be deposited to a greater extent along the pathway leading to the lungs, such as in the upper airways (oropharynx and larynx) and the central airways (trachea and primary and secondary bronchous). Thus, for pMDI systems which have a particle distribution tending toward the large particle size, the percentage of effective API deliver is relatively small since a larger proportion of the API is not delivered to the peripheral airways where it is most effective, i.e., tertiary bronchi, terminal broncioes and alveoli.

WO 2023/039104 discloses a comparison of ipratropium bromide monohydrate formulations and/or anhydrous ipratropium bromide in three propellants, namely, HFC-134a or HFC-152a or HFO-1234ze(E), in combination with ethanol as a cosolvent. This document states that the saturated solubility of ipratropium bromide was significantly lower in HFO-1234ze (E) compositions with 15% ethanol when compared to similar solutions in HFA-134a and HFA-152a.

WO2023/039103 mentions that HFOs have been proposed as propellants for MDIs but also notes that no MDI product has been successfully developed or commercialized using HFOs as a propellant. The '103 publication discloses an MDI that uses a formulation comprising greater than 70% by weight of HFO-1234ze(E), ethanol and at least one active pharmaceutical ingredient (API). The amounts of ethanol disclosed as being used in the '103 formulations range from as low as 0.1 wt. % to as high as 20%.

It is not generally predictable how a change in propellant affects the mass or particle size distribution of medication delivered from pMDIs, but it is nevertheless highly desirable to provide pMDIs that use low GWP propellant, especially instead of HFC-134a. For this reason, it has been a significant technical challenge to deliver even the same effective mass of medication per actuation achieved using HFC-134a as the propellant.

Applicants have developed pMDIs for ipratropium that provide not only a low GWP alternative to HFC-134a pMDIs but which also are unexpectedly able to dramatically increase the effective dose per actuation, as described in detail hereinafter.

### SUMMARY

The present invention includes methods for delivering a dose of ipratropium bromide comprising:
a. providing in a pMDI comprising:
   i. a canister;
   ii. a formulation comprising ipratropium bromide in solution contained in the canister, said formulation comprising: (1) from about 75% by weight to about 88% by weight of HFO-1234ze(E); (2) from about 12% by weight to about 25% by weight of ethanol; and (3) ipratropium bromide; and
   iii. a metering valve releasably closing the canister and fluidly connected to an actuator having an orifice diameter of less than 0.5 mm; and
b. actuating said pMDI to produce an aerosol spray of said ipratropium bromide.
   For the purposes of convenience, methods according to this paragraph are referred to as Pharmaceutical Delivery Method 1A1.

The present invention includes methods for delivering a dose of ipratropium bromide comprising:
a. providing in a pMDI comprising:
   i. a canister;
   ii. a formulation comprising ipratropium bromide in solution contained in the canister, said formulation comprising: (1) from about 75% by weight to about 88% by weight of HFO-1234ze(E); (2) from about 12% by weight to about 25% by weight of ethanol; (3) from about 0.1% by weight to about 1.5% by weight of water; and (4) ipratropium bromide; and
   iii. a metering valve releasably closing the canister and fluidly connected to an actuator having an orifice diameter of less than 0.5 mm; and
b. actuating said pMDI to produce an aerosol spray of said ipratropium bromide.
   For the purposes of convenience, methods according to this paragraph are referred to as Pharmaceutical Delivery Method 1A2.

The present invention includes methods for delivering a dose of ipratropium bromide comprising:
a. providing in a pMDI comprising:
   iv. a canister;
   v. a formulation comprising ipratropium bromide in solution contained in the canister, said formulation comprising: (1) from about 82% by weight to about 87% by weight of HFO-1234ze(E); (2) from about 13% by weight to about 18% by weight of ethanol; and (3) ipratropium bromide; and
   vi. a metering valve releasably closing the canister and fluidly connected to an actuator having an orifice diameter of less than 0.5 mm; and
c. actuating said pMDI to produce an aerosol spray comprising said ipratropium bromide.
   For the purposes of convenience, methods according to this paragraph are referred to as Pharmaceutical Delivery Method 1B1.

The present invention includes methods for delivering a dose of ipratropium bromide comprising:
a. providing in a pMDI comprising:
   i. a canister;
   ii. a formulation comprising ipratropium bromide in solution contained in the canister, said formulation comprising: (1) from about 82% by weight to about 87% by weight of HFO-1234ze(E); (2) from about 13% by weight to about 18% by weight of ethanol; (3) from about 0.1% by weight to about 1.5% by weight of water; and (4) ipratropium bromide; and
   iii. a metering valve releasably closing the canister and fluidly connected to an actuator having an orifice diameter of less than 0.5 mm; and
d. actuating said pMDI to produce an aerosol spray comprising said ipratropium bromide.
   For the purposes of convenience, methods according to this paragraph are referred to as Pharmaceutical Delivery Method 1B2.

The present invention includes methods for delivering a dose of ipratropium bromide comprising:
a. providing in a pMDI comprising:
   i. a canister;
   ii. a formulation comprising ipratropium bromide in solution contained in the canister, said formulation comprising: (1) from about 82% by weight to about 86% by weight of HFO-1234ze(E); (2) from about 14% by weight to about 18% by weight of ethanol; and (3) ipratropium bromide; and
   iii. a metering valve releasably closing the canister and having an orifice with an outside diameter of less than 0.5 mm; and
b. actuating said pMDI to produce an aerosol spray comprising said ipratropium bromide.
   For the purposes of convenience, methods according to this paragraph are referred to as Pharmaceutical Delivery Method 1C1.

The present invention includes methods for delivering a dose of ipratropium bromide comprising:
a. providing in a pMDI comprising:
   i. a canister;
   ii. a formulation comprising ipratropium bromide in solution contained in the canister, said formulation comprising: (1) from about 82% by weight to about 86% by weight of HFO-1234ze(E); (2) from about 14% by weight to about 18% by weight of ethanol; (3) from about 0.1% by weight to about 1.5% by weight of water; and (4) ipratropium bromide; and
   iii. a metering valve releasably closing the canister and having an orifice with an outside diameter of less than 0.5 mm; and
b. actuating said pMDI to produce an aerosol spray comprising said ipratropium bromide.
   For the purposes of convenience, methods according to this paragraph are referred to as Pharmaceutical Delivery Method 1C2.

The present invention includes methods for delivering a dose of ipratropium bromide comprising:
a. providing in a pMDI comprising:
   a. a canister;
   b. a formulation comprising ipratropium bromide in solution contained in the canister, said formulation comprising: (1) from about 80% by weight to about 88% by weight of HFO-1234ze(E); (2) from about 12% by weight to about 20% by weight of ethanol; (3) from about 0.1% by weight to about 1.5% by weight of water; and (3) ipratropium bromide; and
   c. a metering valve releasably closing the canister and having an orifice with an outside diameter of less than 0.5 mm; and
b. actuating said pMDI to produce an aerosol spray comprising said ipratropium bromide, wherein said aerosol spray has a fine particle fraction of greater than about 40%.
   For the purposes of convenience, methods according to this paragraph are referred to as Pharmaceutical Delivery Method 1D1.

The present invention includes methods for delivering a dose of ipratropium bromide comprising:
a. providing in a pMDI comprising:
   i. a canister;
   ii. a formulation comprising ipratropium bromide in solution contained in the canister, said formulation comprising: (1) from about 80% by weight to about 88% by weight of HFO-1234ze(E); (2) from about 12% by weight to about 20% by weight of ethanol; (3) from about 0.1% by weight to about 1.5% by weight of water; and (4) ipratropium bromide; and
   iii. a metering valve releasably closing the canister and having an orifice with an outside diameter of less than 0.5 mm; and
b. actuating said pMDI to produce an aerosol spray comprising said ipratropium bromide, wherein said aerosol spray has a fine particle fraction of greater than about 40%.
   For the purposes of convenience, methods according to this paragraph are referred to as Pharmaceutical Delivery Method 1D2.

The present invention includes methods for delivering a dose of ipratropium bromide comprising:
a. providing in a pMDI comprising:
   i. a canister;
   ii. a formulation comprising ipratropium bromide in solution contained in the canister, said formulation comprising: (1) from about 80% by weight to about 88% by weight of HFO-1234ze(E); (2) from about 12% by weight to about 20% by weight of ethanol; and (3) ipratropium bromide; and
   iii. a metering valve releasably closing the canister and fluidly connected to an actuator having an orifice diameter of less than 0.4 mm; and
b. actuating said pMDI to produce an aerosol spray comprising said ipratropium bromide, wherein said aerosol spray has a fine particle fraction of greater than about 45%.
   For the purposes of convenience, methods according to this paragraph are referred to as Pharmaceutical Delivery Method 1E1.

The present invention includes methods for delivering a dose of ipratropium bromide comprising:
a. providing in a pMDI comprising:
   i. a canister;
   ii. a formulation comprising ipratropium bromide in solution contained in the canister, said formulation comprising: (1) from about 80% by weight to about 88% by weight of HFO-1234ze(E); (2) from about 12% by weight to about 20% by weight of ethanol; (3) from about 0.1% by weight to about 1.5% by weight of water; and (4) ipratropium bromide; and
   iii. a metering valve releasably closing the canister and fluidly connected to an actuator having an orifice diameter of less than 0.4 mm; and
b. actuating said pMDI to produce an aerosol spray comprising said ipratropium bromide, wherein said aerosol spray has a fine particle fraction of greater than about 45%.
   For the purposes of convenience, methods according to this paragraph are referred to as Pharmaceutical Delivery Method 1E2.

The present invention also includes a pressurized metered dose inhaler (pMDI) comprising:
a. a container;
b. a pharmaceutical formulation under pressure in the container, said pharmaceutical formulation comprising: (i) about from about 80% by weight to about 88% by weight of HFO-1234ze(E); (ii) from about 12% by weight to about 20% by weight of ethanol; and (iii) ipratropium bromide in solution in said HFO-1234ze and/or said ethanol; and
c. a normally closed valve on the container,
said valve being fluidly connected to an actuator having an orifice diameter of less than 0.5 mm, said normally closed valve being actuatable to an open position to release an aerosol spray containing ipratropium bromide in solution from said container.
   For the purposes of convenience, pMDIs according to this paragraph are referred to as MDI1A1.

The present invention also includes a pressurized metered dose inhaler (pMDI) comprising:
a. a container;
b. a pharmaceutical formulation under pressure in the container, said pharmaceutical formulation comprising: (i) about from about 80% by weight to about 88% by weight of HFO-1234ze(E); (ii) from about 12% by weight to about 20% by weight of ethanol; (iii) from about 0.1% by weight to about 1.5% by weight of water; and (iv) ipratropium bromide in solution in said HFO-1234ze and/or said ethanol; and
c. a normally closed valve on the container,
said valve being fluidly connected to an actuator having an orifice diameter of less than 0.5 mm, said normally closed valve being actuatable to an open position to release an aerosol spray containing ipratropium bromide in solution from said container.
   For the purposes of convenience, pMDIs according to this paragraph are referred to as MDI1A2.

The present invention also includes a pressurized metered dose inhaler (pMDI) comprising:
a. a container;
b. a pharmaceutical formulation under pressure in the container, said pharmaceutical formulation comprising: (i) about from about 80% by weight to about 88% by weight of HFO-1234ze(E); (ii) from about 12% by weight to about 20% by weight of ethanol; and (iii) ipratropium bromide in solution in said HFO-1234ze and/or said ethanol; and
c. a normally closed valve on the container and being fluidly connected to an actuator having an orifice diameter of less than 0.45 mm, said valve being actuatable to an open position to release an aerosol spray containing ipratropium bromide in solution from said container.
   For the purposes of convenience, pMDIs according to this paragraph are referred to as MDI1B1.

The present invention also includes a pressurized metered dose inhaler (pMDI) comprising:
a. a container;
b. a pharmaceutical formulation under pressure in the container, said pharmaceutical formulation comprising: (i) about from about 80% by weight to about 88% by weight of HFO-1234ze(E); (ii) from about 12% by weight to about 20% by weight of ethanol; (iii) from about 0.1% by weight to about 1.5% by weight of water; and (iv) ipratropium bromide in solution in said HFO-1234ze and/or said ethanol; and
c. a normally closed valve on the container and being fluidly connected to an actuator having an orifice diameter of less than 0.45 mm, said valve being actuatable to an open position to release an aerosol spray containing ipratropium bromide in solution from said container.
   For the purposes of convenience, pMDIs according to this paragraph are referred to as MDI1B2.

The present invention also includes a pressurized metered dose inhaler (pMDI) comprising:
a. a container;
b. a pharmaceutical formulation under pressure in the container, said pharmaceutical formulation comprising: (i) about from about 80% by weight to about 88% by weight of HFO-1234ze(E); (ii) from about 12% by weight to about 20% by weight of ethanol; and (iii) ipratropium bromide in solution in said HFO-1234ze and/or said ethanol; and
c. a normally closed valve on the container and being fluidly connected to an actuator having an orifice diameter of from about 0.2 mm to less than 0.4 mm, said valve being actuatable to an open position to release an aerosol spray containing ipratropium bromide in solution from said container.
   For the purposes of convenience, pMDIs according to this paragraph are referred to as MDI1C1.

The present invention also includes a pressurized metered dose inhaler (pMDI) comprising:
a. a container;
b. a pharmaceutical formulation under pressure in the container, said pharmaceutical formulation comprising: (i) about from about 80% by weight to about 88% by weight of HFO-1234ze(E); (ii) from about 12% by weight to about 20% by weight of ethanol; (iii) from about 0.1% by weight to about 1.5% by weight of water; and (iv) ipratropium bromide in solution in said HFO-1234ze and/or said ethanol; and
c. a normally closed valve on the container and being fluidly connected to an actuator having an orifice diameter of from about 0.2 mm to less than 0.4 mm, said valve being actuatable to an open position to release an aerosol spray containing ipratropium bromide in solution from said container.
   For the purposes of convenience, pMDIs according to this paragraph are referred to as MDI1C2.

The present invention also includes a pressurized metered dose inhaler (pMDI) comprising:
a. a container;
b. a pharmaceutical formulation under pressure in the container, said pharmaceutical formulation comprising: (i) about from about 85% by weight to about 95% by weight of HFO-1234ze(E); (ii) from about 5% by weight to about 15% by weight of ethanol; and (iii) ipratropium bromide in solution in said HFO-1234ze and/or said ethanol; and
c. a normally closed valve on the container fluidly connected to an actuator having an orifice diameter of from about 0.2 mm to less than 0.3 mm, said valve being actuatable to an open position to release an aerosol spray containing ipratropium bromide in solution from said container.
   For the purposes of convenience, pMDIs according to this paragraph are referred to as MDI1D1.

The present invention also includes a pressurized metered dose inhaler (pMDI) comprising:
a. a container;
b. a pharmaceutical formulation under pressure in the container, said pharmaceutical formulation comprising: (i) about from about 85% by weight to about 95% by weight of HFO-1234ze(E); (ii) from about 5% by weight to about 15% by weight of ethanol; (iii) from about 0.1% by weight to about 1.5% by weight of water; and (iv) ipratropium bromide in solution in said HFO-1234ze and/or said ethanol; and
c. a normally closed valve on the container fluidly connected to an actuator having an orifice diameter of from about 0.2 mm to less than 0.3 mm, said valve being actuatable to an open position to release an aerosol spray containing ipratropium bromide in solution from said container.
   For the purposes of convenience, pMDIs according to this paragraph are referred to as MDI1D2.

The present invention includes pharmaceutical compositions in the form of an aerosol spray comprising particles comprising ipratropium bromide and wherein greater than about 40% by weight of said spray of particles are particles having a size of 5 microns or less. For the purposes of convenience, pharmaceutical compositions according to this paragraph are referred to as Pharmaceutical Composition 1.

The present invention includes pharmaceutical compositions in the form of an aerosol spray comprising particles comprising ipratropium bromide and wherein greater than about 45% by weight of said spray of particles are particles having a size of 5 microns or less. For the purposes of convenience, pharmaceutical compositions according to this paragraph are referred to as Pharmaceutical Composition 2.

The present invention includes pharmaceutical compositions in the form of an aerosol spray comprising particles comprising ipratropium bromide and wherein greater than about 50% by weight of said spray of particles are particles having a size of 5 microns or less. For the purposes of convenience, pharmaceutical compositions according to this paragraph are referred to as Pharmaceutical Composition 3.

### BRIEF DESCRIPTION OF THE DRAWINGS

The invention will now be described with reference to the accompanying drawings in which:

FIG. 1 is a cross-sectional side view of an inhaler including a canister containing a valve and an actuator according to the present disclosure.

FIG. 2 is a detailed cross-sectional side view of the inhaler of FIG. 1.

FIG. 3 is a cross-sectional side view of a metering valve for an inhaler.

FIG. 4 is photographic and schematic depiction of the collection apparatus described in Comparative Example 2A-2B.

FIG. 5 is a chart of the data from Comparative Example 2A-2B.

FIG. 6 is a chart of the data from Example 2A.

### DETAILED DESCRIPTION OF PREFERRED EMBODIMENTS

### I. Definitions

For the purposes of this invention, the term “about” in relation to the amounts expressed in weight percent means that the amount of the component can vary by an amount of +/−10% on a relative basis by weight. Thus, for example, an amount specified as “about 10%” means the amount can vary from 9% to 11%, and amount specified as about 5% can vary from 4.5% to 5.5%.

For the purposes of this invention, the terms “composition” and formulation are used in the broad sense to include both single phase compositions and compositions that comprise two or more phases, such as for example may exist inside a pMDI canister or in an aerosol spray.

The terms “pharmaceutical composition” and “pharmaceutical formulation” are used herein to include any composition or formulation which comprises at least one agent, ingredient, drug, compound, composition, or other substance that may be used on, or administered to, a human or animal for a purpose that includes one or more of therapeutic, pharmaceutical, pharmacological, diagnostic, and prophylactic and immunomodulation.

The term “delivered dose” and its abbreviation “DD” refers to the amount of ipratropium contained in the volume of composition that exits the actuator nozzle of a pMDI.

As used herein, the term “carrier” refers to one or more pharmacologically inert substances which provide a continuous phase in which ipratropium (including ipratropium bromide) is solvated and/or carried and which comprise components that exert a sufficiently high vapor pressure at normal room temperature to propel the particles from the canister of a pMDI to a patient upon actuation of the MDI's metering valve. Therefore, the term “carrier” encompasses both a single component and a combination of two or more different components that form the medium in which the ipratropium is solvated or otherwise carried. Thus, the HFO-1234ze(E) component of the carrier of present composition acts at least as a propellent.

The term “respirable” generally refers to particles, aggregates, drops, etc. sized such that they can be inhaled and reach the airways of the lung, and is generally understood to refer to particles having a size of about 5 microns or less.

The terms “HFC-134a” and “134a” each mean 1,1,1,2-tetrafluoroethane.

The terms “HFO-1234ze(E),” and “1234ze(E)” as used herein each mean trans-1,3,3,3-tetrafluoropropene. Unless otherwise stated, “HFO-1234ze” and “1234ze” mean trans-1,3,3,3-tetrafluoropropene.

The term “ipratropium” as used herein encompasses any and all pharmaceutically acceptable versions of ipratropium, including pharmaceutically acceptable salts of ipratropium (such as ipratropium bromide).

Reference herein to a group of defined items includes all such defined items, including all such items with suffix designations. Thus, for example, a reference herein to “MDI1” is a specific reference to each of MDI1A, MDI1B, MDI1C, MDI1D and MDI1E.

### II. The Compositions

The preferred pharmaceutical compositions of the present invention, including each of Pharmaceutical Compositions 1-3, preferably comprise solutions of ipratropium in one or more components of the carrier, including particularly one or both of HFO-1234ze(E) and ethanol.

The preferred pharmaceutical compositions of the present invention, including each of Pharmaceutical Compositions 1-3, are physically stable. The preferred pharmaceutical compositions of the present invention, including each of Pharmaceutical Compositions 1, are chemically stable. The preferred pharmaceutical compositions of the present invention, including each of Pharmaceutical Compositions 1-3, are physically stable and chemically stable.

The concentration of the components in the present compositions can generally vary widely within the broad scope of the present invention.

For all compositions of the present invention, other than those defined as “consisting of” the designated components, including each of Pharmaceutical Compositions 1, additional components or excipients may be present. These components may have various uses and functions, including, but not limited to, facilitating formation of the solution, stabilizing the solution, and/or aiding in chemical stabilization of ipratropium or other components.

Preferred excipients include are suitable for inhaled delivery and do not substantially degrade the solution.

For all compositions of the present invention, including each of Pharmaceutical Compositions 1-3, the composition comprises a solution of the indicated ipratropium in the HFO-1234ze(E) and/or ethanol. As used herein, the term solution means that essentially all of the ipratropium is in solution.

In certain preferred forms, the compositions of the present invention, including each of Pharmaceutical Compositions 1-3, have a Global Warming Potential (GWP) of not greater than about 300, more preferably not greater than about 150, not greater than 75, and most preferably not greater than about 10. As used herein, “GWP” is measured relative to that of carbon dioxide and over a 100-year time horizon, as defined in “The Scientific Assessment of Ozone Depletion, 2002, a report of the World Meteorological Association's Global Ozone Research and Monitoring Project,” which is incorporated herein by reference.

In certain preferred forms, the present compositions also preferably have an Ozone Depletion Potential (ODP) of not greater than 0.05, more preferably not greater than 0.02 and even more preferably about zero. As used herein, “ODP” is as defined in “The Scientific Assessment of Ozone Depletion, 2002, A report of the World Meteorological Association's Global Ozone Research and Monitoring Project,” which is incorporated herein by reference.

The composition or formulation of the present of the present invention contained in the canister of the present pMDIs, including each of MDI1A through MDI1D, comprises from about 80% to about 88% by weight of HFO-1234ze(E), from about 12% to about 20% of ethanol, and from about 0.01 wt % about 0.06 wt % of ipratropium. Particular formulations to be included in the present pMDIs, including each of MDI1A through MDI1D, include those identified in the following Table PF for “pharmaceutical formulation,” wherein the first column of the table includes “PF” as an abbreviation for a defined Pharmaceutical Formulation. In Table 1 below: “IP-AS” means any pharmaceutically effective salt of ipratropium; “IB” means ipratropium bromide, whether added to the formulation as anhydrous ipratropium bromide or as ipratropium bromide monohydrate; “NR” means that the component or an particular amount is “not required” according to the specified PF definition and as such its presence in any amount or in no amount is permitted; “Yes” means the component is required but that any type or amount is permitted; “Comp” means that the specified pharmaceutical composition comprises the items identified in the table; “CEO” means that the specified composition consists essentially of the items identified in the table; “CO” means that composition consists of the items identified in the table; the column heading Excipient indicates the pharmaceutical composition comprises the type or category of excipient as defined herein; the column heading Excipient Amount indicates the amount of the indicated excipient as a percentage of the pharmaceutical composition; and all amounts are understood to be preceded by the word “about.”

TABLE PC

| | | Carrier components, | | | | | Excipient | |
|---|---|---|---|---|---|---|---|---|
| | | wt % of PC | | | Ipratropium | | | Amount, |
| | | 1234ze | | | Type or | | Type or | wt % of |
| PC No. | | (E) | Ethanol | Water | specific | wt % of PC | specific | PC |
| 4A | Comp | 80-<89 | >10-20 | NR | IP-AS | 0.01-0.06 | NR | NR |
| 4B | CEO | 80-<89 | >11-20 | NF | IP-AS | 0.01-0.06 | NR | NR |
| 4C | Comp | 80-88 | 12-20 | NR | IP-AS | 0.01-0.06 | NR | NR |
| 4D | CEO | 80-87 | 13-20 | NR | IP-AS | 0.01-0.06 | NR | NR |
| 4E | Comp | 80-86 | 14-20 | NR | IP-AS | 0.01-0.06 | NR | NR |
| 4F | CEO | 82-87 | 13-18 | NR | IP-AS | 0.01-0.06 | NR | NP |
| 4G | Comp | 83-86 | 14-17 | NR | IP-AS | 0.01-0.06 | NR | NR |
| 4H | CEO | 84-86 | 14-16 | NR | IP-AS | 0.01-0.06 | NR | NR |
| 4I | Comp | 80-<89 | >10-20 | 0.1-1 | IP-AS | 0.01-0.06 | NR | NR |
| 4J | CEO | 80-<89 | >11-20 | 0.1-1 | IP-AS | 0.01-0.06 | NR | NR |
| 4K | Comp | 80-88 | 12-20 | 0.1-1 | IP-AS | 0.01-0.06 | NR | NR |
| 4L | CEO | 80-87 | 13-20 | 0.1-1 | IP-AS | 0.01-0.06 | NR | NR |
| 4M | CEO | 80-86 | 14-20 | 0.1-1 | IP-AS | 0.01-0.06 | NR | NR |
| 4N | Comp | 82-87 | 13-18 | 0.1-1 | IP-AS | 0.01-0.06 | NR | NR |
| 4O | CEO | 83-86 | 14-17 | 0.1-1 | IP-AS | 0.01-0.06 | NR | NR |
| 4P | Comp | 84-86 | 14-16 | 0.1-1 | IP-AS | 0.01-0.06 | NR | NR |
| 4Q | CEO | 82-85 | 15-18 | 0.1-1 | IP-AS | 0.01-0.06 | NR | NR |
| 4R | Comp | 83-85 | 15-17 | 0.1-1 | IP-AS | 0.01-0.06 | NR | NR |
| 4S | CEO | 84 | 16 | 0.1-1 | IP-AS | 0.01-0.06 | NR | NR |
| 4T | CEO | 85 | 15 | 0.1-1 | IP-AS | 0.01-0.06 | NR | NR |
| 4U | CEO | 86 | 14 | 0.1-1 | IP-AS | 0.01-0.06 | NR | NR |
| 5A | Comp | 80-<89 | >10-20 | 0.1-1 | IP-AS | 0.03-0.04 | NR | NR |
| 5B | CEO | 80-<89 | >11-20 | 0.1-1 | IP-AS | 0.03-0.04 | NR | NR |
| 5C | Comp | 80-88 | 12-20 | 0.1-1 | IP-AS | 0.03-0.04 | NR | NR |
| 5D | CEO | 80-87 | 13-20 | 0.1-1 | IP-AS | 0.03-0.04 | NR | NR |
| 5E | Comp | 80-86 | 14-20 | 0.1-1 | IP-AS | 0.03-0.04 | NR | NR |
| 5F | CEO | 82-87 | 13-18 | 0.1-1 | IP-AS | 0.03-0.04 | NR | NR |
| 5G | Comp | 83-86 | 14-17 | 0.1-1 | IP-AS | 0.03-0.04 | NR | NR |
| 5H | CEO | 84-86 | 14-16 | 0.1-1 | IP-AS | 0.03-0.04 | NR | NR |
| 5I | Comp | 80-<89 | >10-20 | 0.1-1 | IP-AS | 0.03-0.04 | NR | NR |
| 5J | CEO | 80-<89 | >11-20 | 0.1-1 | IP-AS | 0.03-0.04 | NR | NR |
| 5K | Comp | 80-88 | 12-20 | 0.1-1 | IP-AS | 0.03-0.04 | NR | NR |
| 5L | CEO | 80-87 | 13-20 | 0.1-1 | IP-AS | 0.03-0.04 | NR | NR |
| 5M | CEO | 80-86 | 14-20 | 0.1-1 | IP-AS | 0.03-0.04 | NR | NR |
| 5N | Comp | 82-87 | 13-18 | 0.1-1 | IP-AS | 0.03-0.04 | NR | NR |
| 5O | CEO | 83-86 | 14-17 | 0.1-1 | IP-AS | 0.03-0.04 | NR | NR |
| 5P | Comp | 84-86 | 14-16 | 0.1-1 | IP-AS | 0.03-0.04 | NR | NR |
| 5Q | CEO | 82-85 | 15-18 | 0.1-1 | IP-AS | 0.03-0.04 | NR | NR |
| 5R | Comp | 83-85 | 15-17 | 0.1-1 | IP-AS | 0.03-0.04 | NR | NR |
| 5S | CEO | 84 | 16 | 0.1-1 | IP-AS | 0.03-0.04 | NP | NR |
| 5T | CEO | 85 | 15 | 0.1-1 | IP-AS | 0.03-0.04 | NR | NR |
| 5U | CEO | 86 | 14 | 0.1-1 | IP-AS | 0.03-0.04 | NR | NR |
| 6A | Comp | 80-<89 | >10-20 | 0.1-1 | IB | 0.03-0.04 | Citric acid | NR |
| 6B | CEO | 80-<89 | >11-20 | 0.1-1 | IB | 0.03-0.04 | Citric acid | NR |
| 6C | Comp | 80-88 | 12-20 | 0.1-1 | IB | 0.03-0.04 | Citric acid | NR |
| 6D | CEO | 80-87 | 13-20 | 0.1-1 | IB | 0.03-0.04 | Citric acid | NR |
| 6E | Comp | 80-86 | 14-20 | 0.1-1 | IB | 0.03-0.04 | Citric acid | NR |
| 6F | CEO | 82-87 | 13-18 | 0.1-1 | IB | 0.03-0.04 | Citric acid | NR |
| 6G | Comp | 83-86 | 14-17 | 0.1-1 | IB | 0.03-0.04 | Citric acid | NR |
| 6H | CEO | 84-86 | 14-16 | 0.1-1 | IB | 0.03-0.04 | Citric acid | NR |
| 6I | Comp | 80-<89 | >10-20 | 0.1-1 | IB | 0.03-0.04 | Citric acid | NR |
| 6J | CEO | 80-<89 | >11-20 | 0.1-1 | IB | 0.03-0.04 | Citric acid | NR |
| 6K | Comp | 80-88 | 12-20 | 0.1-1 | IB | 0.03-0.04 | Citric acid | NR |
| 6L | CEO | 80-87 | 13-20 | 0.1-1 | IB | 0.03-0.04 | Citric acid | NR |
| 6M | CEO | 80-86 | 14-20 | 0.1-1 | IB | 0.03-0.04 | Citric acid | NR |
| 6N | Comp | 82-87 | 13-18 | 0.1-1 | IB | 0.03-0.04 | Citric acid | NR |
| 6O | CEO | 83-86 | 14-17 | 0.1-1 | IB | 0.03-0.04 | Citric acid | NR |
| 6P | Comp | 84-86 | 14-16 | 0.1-1 | IB | 0.03-0.04 | Citric acid | NR |
| 6Q | CEO | 82-85 | 15-18 | 0.1-1 | IB | 0.03-0.04 | Citric acid | NR |
| 6R | Comp | 83-85 | 15-17 | 0.1-1 | IB | 0.03-0.04 | Citric acid | NR |
| 6S | CEO | 84 | 16 | 0.1-1 | IB | 0.03-0.04 | Citric acid | 0.005- |
| | | | | | | | | 0.015 |
| 6T | CEO | 85 | 15 | 0.1-1 | IB | 0.03-0.04 | Citric acid | 0.005- |
| | | | | | | | | 0.015 |
| 6U | CEO | 86 | 14 | 0.1-1 | IB | 0.03-0.04 | Citric acid | 0.005- |
| | | | | | | | | 0.015 |
| 7A | Comp | 80-<89 | >10-20 | NR | IB | 0.01-0.06 | NR | NR |
| 7B | CEO | 80-<89 | >11-20 | NR | IB | 0.01-0.06 | NR | NR |
| 7C | Comp | 80-88 | 12-20 | NR | IB | 0.01-0.06 | NR | NR |
| 7D | CEO | 80-87 | 13-20 | NR | IB | 0.01-0.06 | NR | NR |
| 7E | Comp | 80-86 | 14-20 | NR | IB | 0.01-0.06 | NR | NR |
| 7F | CEO | 82-87 | 13-18 | NR | IB | 0.01-0.06 | NP | NR |
| 7G | Comp | 83-86 | 14-17 | NR | IB | 0.01-0.06 | NR | NR |
| 7H | CEO | 84-86 | 14-16 | NR | IB | 0.01-0.06 | NR | NR |
| 7I | Comp | 80-<89 | >10-20 | 0.1-1 | IB | 0.01-0.06 | NR | NR |
| 7J | CEO | 80-<89 | >11-20 | 0.1-1 | IB | 0.01-0.06 | NF | NR |
| 7K | Comp | 80-88 | 12-20 | 0.1-1 | IB | 0.01-0.06 | NR | NR |
| 7L | CEO | 80-87 | 13-20 | 0.1-1 | IB | 0.01-0.06 | NR | NR |
| 7M | CEO | 80-86 | 14-20 | 0.1-1 | IB | 0.01-0.06 | NR | NR |
| 7N | Comp | 82-87 | 13-18 | 0.1-1 | IB | 0.01-0.06 | NR | NR |
| 7O | CEO | 83-86 | 14-17 | 0.1-1 | IB | 0.01-0.06 | NR | NR |
| 7P | Comp | 84-86 | 14-16 | 0.1-1 | IB | 0.01-0.06 | NR | NR |
| 7Q | CEO | 82-85 | 15-18 | 0.1-1 | IB | 0.01-0.06 | NR | NR |
| 7R | Comp | 83-85 | 15-17 | 0.1-1 | IB | 0.01-0.06 | NR | NR |
| 7S | CEO | 84 | 16 | 0.1-1 | IB | 0.01-0.06 | NR | NR |
| 7T | CEO | 85 | 15 | 0.1-1 | IB | 0.01-0.06 | NR | NR |
| 7U | CEO | 86 | 14 | 0.1-1 | IB | 0.01-0.06 | NR | NR |
| 8A | Comp | 80-<89 | >10-20 | 0.1-1 | IB | 0.03-0.04 | NR | NR |
| 8B | CEO | 80-<89 | >11-20 | 0.1-1 | IB | 0.03-0.04 | NR | NR |
| 8C | Comp | 80-88 | 12-20 | 0.1-1 | IB | 0.03-0.04 | NR | NR |
| 8D | CEO | 80-87 | 13-20 | 0.1-1 | IB | 0.03-0.04 | NR | NR |
| 8E | Comp | 80-86 | 14-20 | 0.1-1 | IB | 0.03-0.04 | NR | NR |
| 8F | CEO | 82-87 | 13-18 | 0.1-1 | IB | 0.03-0.04 | NR | NR |
| 8G | Comp | 83-86 | 14-17 | 0.1-1 | IB | 0.03-0.04 | NR | NR |
| 8H | CEO | 84-86 | 14-16 | 0.1-1 | IB | 0.03-0.04 | NR | NR |
| 8I | Comp | 80-<89 | >10-20 | 0.1-1 | IB | 0.03-0.04 | NR | NR |
| 8J | CEO | 80-<89 | >11-20 | 0.1-1 | IB | 0.03-0.04 | NR | NR |
| 8K | Comp | 80-88 | 12-20 | 0.1-1 | IB | 0.03-0.04 | NR | NR |
| 8L | CEO | 80-87 | 13-20 | 0.1-1 | IB | 0.03-0.04 | NR | NR |
| 8M | CEO | 80-86 | 14-20 | 0.1-1 | IB | 0.03-0.04 | NR | NR |
| 8N | Comp | 82-87 | 13-18 | 0.1-1 | IB | 0.03-0.04 | NR | NR |
| 8O | CEO | 83-86 | 14-17 | 0.1-1 | IB | 0.03-0.04 | NR | NR |
| 8P | Comp | 84-86 | 14-16 | 0.1-1 | IB | 0.03-0.04 | NR | NR |
| 8Q | CEO | 82-85 | 15-18 | 0.1-1 | IB | 0.03-0.04 | NR | NR |
| 8R | Comp | 83-85 | 15-17 | 0.1-1 | IB | 0.03-0.04 | NR | NR |
| 8S | CEO | 84 | 16 | 0.1-1 | IB | 0.03-0.04 | NR | NR |
| 8T | CEO | 85 | 15 | 0.1-1 | IB | 0.03-0.04 | NR | NR |
| 8U | CEO | 86 | 14 | 0.1-1 | IB | 0.03-0.04 | NR | NR |
| 9A | Comp | 80-<89 | >10-20 | 0.1-1 | IB | 0.03-0.04 | Citric acid | NR |
| 9B | CEO | 80-<89 | >11-20 | 0.1-1 | IB | 0.03-0.04 | Citric acid | NR |
| 9C | Comp | 80-88 | 12-20 | 0.1-1 | IB | 0.03-0.04 | Citric acid | NR |
| 9D | CEO | 80-87 | 13-20 | 0.1-1 | IB | 0.03-0.04 | Citric acid | NR |
| 9E | Comp | 80-86 | 14-20 | 0.1-1 | IB | 0.03-0.04 | Citric acid | NR |
| 9F | CEO | 82-87 | 13-18 | 0.1-1 | IB | 0.03-0.04 | Citric acid | NR |
| 9G | Comp | 83-86 | 14-17 | 0.1-1 | IB | 0.03-0.04 | Citric acid | NR |
| 9H | CEO | 84-86 | 14-16 | 0.1-1 | IB | 0.03-0.04 | Citric acid | NR |
| 9I | Comp | 80-< | >10-20 | 0.1-1 | IB | 0.03-0.04 | Citric acid | NR |
| 9J | CEO | 80-<89 | >11-20 | 0.1-1 | IB | 0.03-0.04 | Citric acid | NR |
| 9K | Comp | 80-88 | 12-20 | 0.1-1 | IB | 0.03-0.04 | Citric acid | NR |
| 9L | CEO | 80-87 | 13-20 | 0.1-1 | IB | 0.03-0.04 | Citric acid | NR |
| 9M | CEO | 80-86 | 14-20 | 0.1-1 | IB | 0.03-0.04 | Citric acid | NR |
| 9N | Comp | 82-87 | 13-18 | 0.1-1 | IB | 0.03-0.04 | Citric acid | NR |
| 9O | CEO | 83-86 | 14-17 | 0.1-1 | IB | 0.03-0.04 | Citric acid | NR |
| 9P | Comp | 84-86 | 14-16 | 0.1-1 | IB | 0.03-0.04 | Citric acid | NR |
| 9Q | CEO | 82-85 | 15-18 | 0.1-1 | IB | 0.03-0.04 | Citric acid | NR |
| 9R | Comp | 83-85 | 15-17 | 0.1-1 | IB | 0.03-0.04 | Citric acid | NR |
| 9S | CEO | 84 | 16 | 0.1-1 | IB | 0.03-0.04 | Citric acid | 0.005- |
| | | | | | | | | 0.015 |
| 9T | CEO | 85 | 15 | 0.1-1 | IB | 0.03-0.04 | Citric acid | 0.005- |
| | | | | | | | | 0.015 |
| 9U | CEO | 86 | 14 | 0.1-1 | IB | 0.03-0.04 | Citric acid | 0.005- |
| | | | | | | | | 0.015 |

### III. Devices and Methods

The present invention includes devices for the delivery by inhalation of the compositions and formulations of the present invention, including each of MDI1A through MDI1D. In certain preferred embodiments, the devices of the present invention, including each of MDI1A through MDI1D, comprise a container, preferably an aerosol canister, containing a pressurized composition of the present invention and having a metered dose dispensing valve operable between non-dispensing and dispensing positions. The present devices, including each of MDI1A through MDI1D, preferably also comprise an actuator, which in preferred embodiments comprises a housing adapted to receive the aerosol container and to define a chamber in fluid communication with a patient port for introducing the medicament into the oral and/or nasal cavity of the patient, preferably in the form of a mouthpiece and/or nasal adapter. The actuator also preferably includes a nozzle block adapted to receive the valve stem of the dispensing valve, the nozzle block preferably comprising a passage in fluid communication with the valve stem and terminating in an orifice for directing medicament from the valve stem into the chamber.

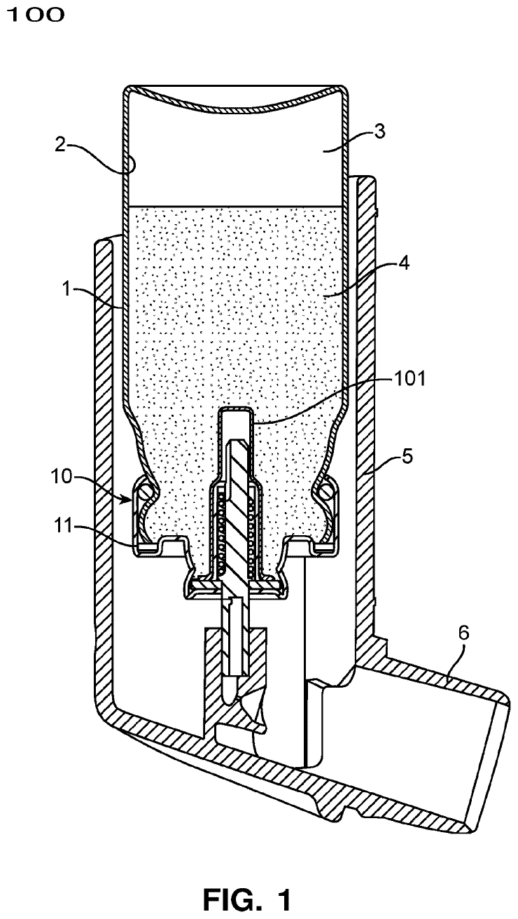

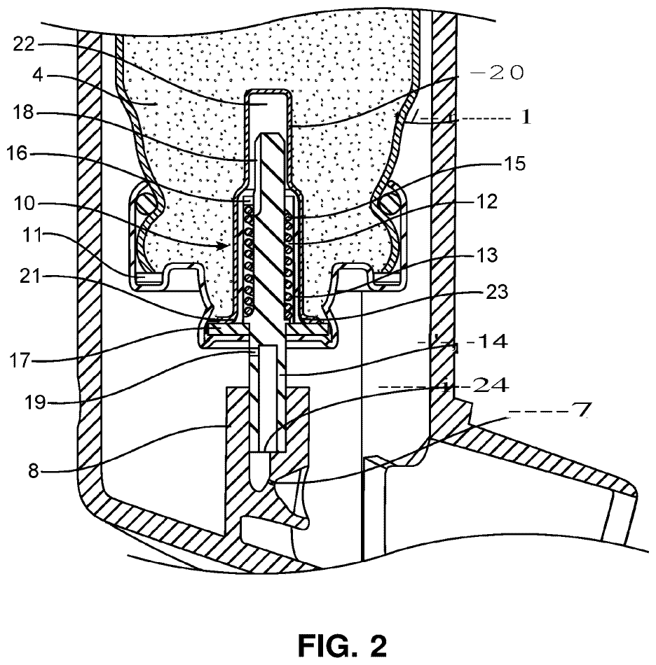

By way of example but not by way of limitation, FIG. 1 shows one embodiment of a metered dose inhaler 100, including an aerosol canister 1 fitted with a metered dose metering valve 10 (shown in its resting position). The metering valve 10 is typically affixed, i.e., crimped, onto the canister via a cap or ferrule 11 (typically made of aluminum or an aluminum alloy) which is generally provided as part of the valve assembly. Between the canister and the ferrule there may be one or more seals. In the embodiments shown in FIG. 1 and FIG. 2 between the canister 1 and the ferrule 11 there are two seals including, e.g., an O-ring seal and a gasket seal.

As shown in FIG. 1, the canister/valve dispenser is typically provided with an actuator 5 including an appropriate patient port 6, such as a mouthpiece. For administration to the nasal cavities the patient port is generally provided in an appropriate form (e.g., smaller diameter tube, often sloping upwardly) for delivery through the nose. Actuators are generally made of a plastic material, for example polypropylene or polyethylene. As can be seen from FIG. 1, inner walls 2 of the canister and outer walls 101 of the portion(s) of the metering valve 10 located within the canister define a formulation chamber 3 in which aerosol composition 4 is contained.

The valve 10 shown in FIG. 1 and FIG. 2, includes a metering chamber 12, defined in part by an inner valve body 13, through which a valve stem 14 passes. The valve stem 14, which is biased outwardly by a compression spring 15, is in sliding sealing engagement with an inner tank seal 16 and an outer diaphragm seal 17. The valve 10 also includes a second valve body 20 in the form of a bottle emptier. The inner valve body 13 (also referred to as the “primary” valve body) defines in part the metering chamber 12. The second valve body 20 (also referred to as the “secondary” valve body) defines in part a pre-metering region or chamber besides serving as a bottle emptier.

Referring to FIG. 2, aerosol composition 4 can pass from the composition chamber 3 into a pre-metering chamber 22 provided between the secondary valve body 20 and the primary valve body 13 through an annular space 21 between a flange 23 of the secondary valve body 20 and the primary valve body 13. To actuate (fire) the valve 10, the valve stem 14 is pushed inwardly relative to the canister 1 from its resting position shown in FIG. 1 and FIG. 2, allowing composition to pass from the metering chamber 12 through a side hole 19 in the valve stem and through a stem outlet 24 to a nozzle block 8, which includes an actuator nozzle comprising an orifice 7 then out to the patient through port 6 (see FIG. 1). When the valve stem 14 is released, composition enters into the valve 10, in particular into the pre-metering chamber 22, through the annular space 21 and thence from the pre-metering chamber through a groove 18 in the valve stem past the tank seal 16 into the metering chamber 12.

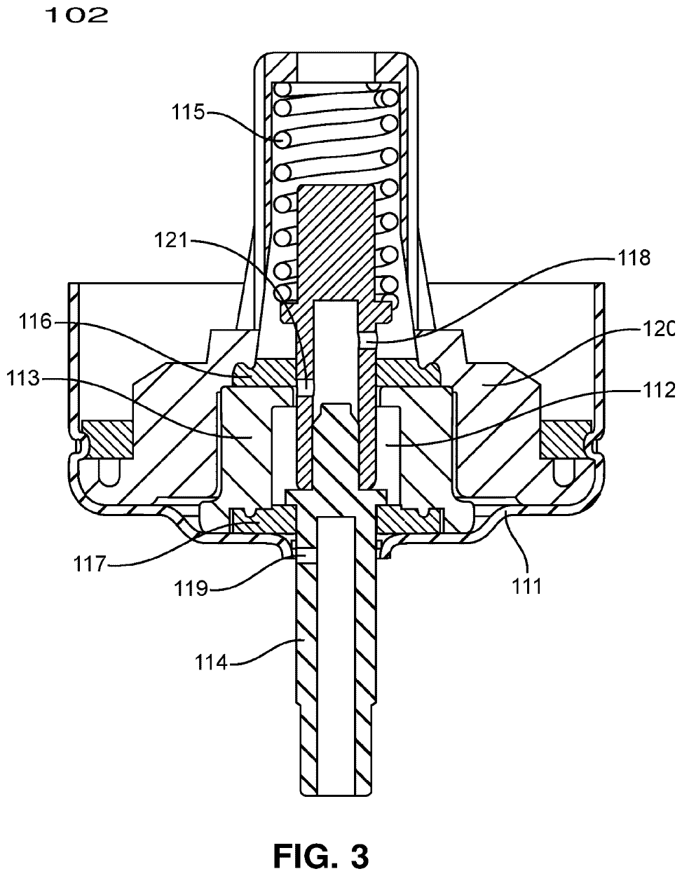

FIG. 3 shows another embodiment of a metered dose aerosol metering valve 102, different from the embodiment shown in FIG. 1 and FIG. 2, in its rest position. The valve 102 has a metering chamber 112 defined in part by a metering tank 113 through which a stem 114 is biased outwardly by spring 115. The stem 114 is made in two parts that are push fit together before being assembled into the valve 102. The stem 114 has an inner seal 116 and an outer seal 117 disposed about it and forming sealing contact with the metering tank 113. A valve body 120 crimped into a ferrule 111 retains the aforementioned components in the valve. In use, composition enters the metering chamber via orifices 121 and 118. The composition's outward path from the metering chamber 112 when a dose is dispensed is via orifice 119.

In certain embodiments the invention, including each of MDI1A through MDI1D, is constructed such that airflow due to patient inhalation is prevented or reduced in the vicinity of the orifice at all times or except during dispensing of the medicament from the valve. Either of such arrangements has the effect of substantially reducing the velocity of the emitted spray compared to an inhaler which allows free flow of air in the vicinity of the nozzle block during dispensing of the medicament.

In certain embodiments, the actuator is constructed such that the distance from the nozzle to the mouthpiece is from approximately 1 to 15 cm, preferably 4 to 6 cm, with a chamber/mouthpiece diameter from 1 to 4 cm, 0.5 to 1 cm in the case of a nasal adapter.

In certain preferred but non-limiting embodiments, the actuator possesses air inlets which enable the patient to inhale though the patient port, preferably without encountering significant resistance since the patient may have breathing difficulties when taking the medication, for example, during an asthma attack. However, the air inlets, for example in the mouthpiece, preferably do not concentrate the airflow into an area that is too narrow, as this will give a high velocity of incoming air which will deflect the spray onto the wall of the mouthpiece opposite the air inlets. In certain preferred embodiments the air inlets are positioned downstream of the nozzle, in the region of the turbulent zone and/or downstream of the turbulent zone. The positioning and direction of the air inlets may also affect the deposition of medicament within the chamber and mouthpiece. In one arrangement air inlets comprise a series of holes and optionally may be interspersed with fluid deflection structures on the wall of the chamber, to direct air into the turbulent zone to mix air with the aerosol stream. Further, the mouthpiece may be constructed of porous material to allow a multiplicity of finely divided air vents to provide air flow over a larger surface area.

In certain embodiments the actuator possesses air inlets upstream of or in the vicinity of the nozzle, but the air inlets are blocked when the valve is fired to release the aerosol spray. The air inlets are opened after the spray has been released by which time the velocity of the stream will have been reduced and the turbulent zone formed. Upon inhalation, an airflow is established from the air inlets to the mouthpiece which entrains the residual aerosol spray. The actuator may include additional air inlets downstream of the nozzle, as described above with respect to the first embodiment. These downstream air inlets do not need to close during release of the aerosol spray.

In certain embodiments, a porous membrane is present to introduce air into or downstream of the turbulent zone. One advantage of the use of such a membrane is that the air is introduced more uniformly and diffusely around the circumference of the spray, thereby acting as a buffer between the turbulent flow and the wall. The effect is to reduce drug deposition in the device. The membrane may optionally be protected from dirt or contact by the user's lips by an additional part of the mouthpiece. When present, it is preferred that the porous membrane material (50) must not significantly impede the patient's ability to inhale through the device. A suitable material is Whatmann No. 4 filter paper; but other materials may be used, such as those used in cylindrical air filters or membrane filters, or such as those formed by sintering polymers. A preferred porous membrane material is in the form of a cylinder made by fusing together small pellets of polypropylene.

For certain medicaments, it is preferred to configure the device so as to reduce contact between the medicament and parts of the patient's body that it is not intended to contact. For example, residues of the medicament deposited on internal surfaces of actuators may be fingered and transferred to other body parts. In such cases, the device may be configured to include one or more fluid flow deflectors to allow the spray to pass through, whilst limiting access by the patient to internal surfaces of the actuator. Of course, the device may be configured for intranasal delivery. This is normally quite undesirable, since the medicaments were designed for delivery to the respiratory system and may not have an appropriate effect when deposited in the oropharynx and allowed to enter the digestive tract. In an effort to overcome this problem, certain embodiments of the present device include the provision of a holding volume, commonly called a spacer, in which the medicament is fired. The spacer preferably allows the velocity of the medicament to be reduced and may also allow some propellant evaporation to occur. Spacers can improve the performance of a metered dose inhaler by reducing oropharyngeal deposition.

The total amount of composition or formulation of the present of the present invention, including each of PC4-PC9, contained in the canister of the present pMDIs, including each of MDI1A through MDI1D, preferably is selected so that at least a portion of the propellant in the canister is present as a liquid after a predetermined number of medicinal doses have been delivered. The predetermined number of doses may be 5 to 200, 30 to 200, 60 to 200, 60 to 120, 60, 120, 200, or any other number of doses. In preferred embodiments, the total amount of composition of the present invention, in the canister may be from 1.0 grams (g) to 30.0 g, 2.0 g to 20.0 g, or 5.0 to 10.0 g. The total amount of formulation, including each of PC4 through PC9 contained in the canister of present pMDIs, including each of MDI1A through MDI1D, is preferably selected to be greater than the product of the predetermined number of doses and the metering volume of the metering valve. In some embodiments, the total amount of composition is greater than 1.1 times, greater than 1.2 times, greater than 1.3 times, greater than 1.4 times, or greater than 1.5 times the product of the predetermined number of doses and the metering volume of the metering valve. This helps to ensure that the amount of each dose remains relatively constant through the life of the inhaler.

The present pMDIs, including each of MDI1A through MDI1D, comprises: a composition of the invention and an actuator orifice having a diameter of greater than 0.15 mm and less than 0.5 mm to produce a fine particle fraction of at least about 40% and a delivered dose of preferably greater than about 20 μg per actuation. Particular MDIs include those identified in the following Table pMDI, wherein: the first column of the table includes “pMDI” as an abbreviation for the pMDI defined in the table; the column headed Pharmaceutical Composition means a pharmaceutical compositions as defined in Table PC above contained in the canister of the pMDI; “NR” means that the component or a particular size “not required” according to the specified pMDI definition and as such its presence in any size is permitted; “Yes” means the component is required but that any type or size is permitted; “Comp” means that the specified pMDI comprises the items identified in the table; and all sizes are understood to be preceded by the word “about.”

TABLE HTC

| | | | | Actuation |
|---|---|---|---|---|
| pMDI, | | Pharmaceutical | Orifice | Property |
| No. | | Composition | Diameter, mm | FPF, % |
| 2A | Comp | 4A | <0.5 | NR |
| 2B | CEO | 4B | <0.5 | NR |
| 2C | Comp | 4C | <0.5 | NR |
| 2D | CEO | 4D | <0.5 | NR |
| 2E | Comp | 4E | <0.5 | NR |
| 2F | CEO | 4F | <0.5 | NR |
| 2G | Comp | 4G | <0.5 | NR |
| 2H | CEO | 4H | <0.5 | NR |
| 2I | Comp | 4I | <0.5 | NR |
| 2J | CEO | 4J | <0.5 | NR |
| 2K | Comp | 4K | <0.5 | NR |
| 2L | CEO | 4L | <0.5 | NR |
| 2M | CEO | 4M | <0.5 | NR |
| 2N | Comp | 4N | <0.5 | NR |
| 2O | CEO | 4O | <0.5 | NR |
| 2P | Comp | 4P | <0.5 | NR |
| 2Q | CEO | 4Q | <0.5 | NR |
| 2R | Comp | 4R | <0.5 | NR |
| 2S | CEO | 4S | <0.5 | NR |
| 2T | CEO | 4T | <0.5 | NR |
| 2U | CEO | 4U | <0.5 | NR |
| 3A | Comp | 5A | <0.5 | NR |
| 3B | CEO | 5B | <0.5 | NR |
| 3C | Comp | 5C | <0.5 | NR |
| 3D | CEO | 5D | <0.5 | NR |
| 3E | Comp | 5E | <0.5 | NR |
| 3F | CEO | 5F | <0.5 | NR |
| 3G | Comp | 5G | <0.5 | NR |
| 3H | CEO | 5H | <0.5 | NR |
| 3I | Comp | 5I | <0.5 | NF |
| 3J | CEO | 5J | <0.5 | NR |
| 3K | Comp | 5K | <0.5 | NR |
| 3L | CEO | 5L | <0.5 | NR |
| 3M | CEO | 5M | <0.5 | NR |
| 3N | Comp | 5N | <0.5 | NR |
| 3O | CEO | 5O | <0.5 | NF |
| 3P | Comp | 5P | <0.5 | NR |
| 3Q | CEO | 5Q | <0.5 | NR |
| 3R | Comp | 5R | <0.5 | NR |
| 3S | CEO | 5S | <0.5 | NR |
| 3T | CEO | 5T | <0.5 | NR |
| 3U | CEO | 5U | <0.5 | NR |
| 4A | Comp | 6A | <0.5 | NP |
| 4B | CEO | 6B | <0.5 | NR |
| 4C | Comp | 6C | <0.5 | NR |
| 4D | CEO | 6D | <0.5 | NR |
| 4E | Comp | 6E | <0.5 | NR |
| 4F | CEO | 6F | <0.5 | NR |
| 4G | Comp | 6G | <0.5 | NR |
| 4H | CEO | 6H | <0.5 | NR |
| 4I | Comp | 6I | <0.5 | NR |
| 4J | CEO | 6J | <0.5 | NR |
| 4K | Comp | 6K | <0.5 | NR |
| 4L | CEO | 6L | <0.5 | NR |
| 4M | CEO | 6M | <0.5 | NF |
| 4N | Comp | 6N | <0.5 | NR |
| 4O | CEO | 6O | <0.5 | NR |
| 4P | Comp | 6P | <0.5 | NR |
| 4Q | CEO | 6Q | <0.5 | NF |
| 4R | Comp | 6R | <0.5 | NR |
| 4S | CEO | 6S | <0.5 | NR |
| 4T | CEO | 6T | <0.5 | NR |
| 4U | CEO | 6U | <0.5 | NR |
| 5A | Comp | 7A | <0.5 | NR |
| 5B | CEO | 7B | <0.5 | NR |
| 5C | Comp | 7C | <0.5 | NR |
| 5D | CEO | 7D | <0.5 | NR |
| 5E | Comp | 7E | <0.5 | NF |
| 5F | CEO | 7F | <0.5 | NR |
| 5G | Comp | 7G | <0.5 | NR |
| 5H | CEO | 7H | <0.5 | NR |
| 5I | Comp | 7I | <0.5 | NR |
| 5J | CEO | 7J | <0.5 | NR |
| 5K | Comp | 7K | <0.5 | NR |
| 5L | CEO | 7L | <0.5 | NR |
| 5M | CEO | 7M | <0.5 | NR |
| 5N | Comp | 7N | <0.5 | NR |
| 5O | CEO | 7O | <0.5 | NR |
| 5P | Comp | 7P | <0.5 | NR |
| 5Q | CEO | 7Q | <0.5 | NR |
| 5R | Comp | 7R | <0.5 | NR |
| 5S | CEO | 7S | <0.5 | NR |
| 5T | CEO | 7T | <0.5 | NR |
| 5U | CEO | 7U | <0.5 | NR |
| 6A | Comp | 8A | <0.5 | NR |
| 6B | CEO | 8B | <0.5 | NR |
| 6C | Comp | 8C | <0.5 | NR |
| 6D | CEO | 8D | <0.5 | NR |
| 6E | Comp | 8E | <0.5 | NR |
| 6F | CEO | 8F | <0.5 | NR |
| 6G | Comp | 8G | <0.5 | NR |
| 6H | CEO | 8H | <0.5 | NR |
| 6I | Comp | 8I | <0.5 | NR |
| 6J | CEO | 8J | <0.5 | NR |
| 6K | Comp | 8K | <0.5 | NR |
| 6L | CEO | 8L | <0.5 | NR |
| 6M | CEO | 8M | <0.5 | NR |
| 6N | Comp | 8N | <0.5 | NR |
| 6O | CEO | 8O | <0.5 | NR |
| 6P | Comp | 8P | <0.5 | NR |
| 6Q | CEO | 8G | <0.5 | NR |
| 6R | Comp | 8R | <0.5 | NR |
| 6S | CEO | 8S | <0.5 | NR |
| 6T | CEO | 8T | <0.5 | NR |
| 6U | CEO | 8U | <0.5 | NR |
| 7A | Comp | 9A | <0.5 | NR |
| 7B | CEO | 9B | <0.5 | NR |
| 7C | Comp | 9C | <0.5 | NR |
| 7D | CEO | 9D | <0.5 | NR |
| 7E | Comp | 9E | <0.5 | NR |
| 7F | CEO | 9F | <0.5 | NR |
| 7G | Comp | 9G | <0.5 | NR |
| 7H | CEO | 9H | <0.5 | NR |
| 7I | Comp | 9I | <0.5 | NR |
| 7J | CEO | 9J | <0.5 | NR |
| 7K | Comp | 9K | <0.5 | NR |
| 7L | CEO | 9L | <0.5 | NR |
| 7M | CEO | 9M | <0.5 | NR |
| 7N | Comp | 9N | <0.5 | NR |
| 7O | CEO | 9O | <0.5 | NR |
| 7P | Comp | 9P | <0.5 | NR |
| 7Q | CEO | 9Q | <0.5 | NR |
| 7R | Comp | 9R | <0.5 | NR |
| 7S | CEO | 9S | <0.5 | NR |
| 7T | CEO | 9T | <0.5 | NR |
| 7U | CEO | 9U | <0.5 | NR |
| 8A | Comp | 4A | >0.15-<0.45 | NR |
| 8B | CEO | 4B | >0.15-<0.45 | NR |
| 8C | Comp | 4C | >0.15-<0.45 | NR |
| 8D | CEO | 4D | >0.15-<0.45 | NR |
| 8E | Comp | 4E | >0.15-<0.45 | NR |
| 8F | CEO | 4F | >0.15-<0.45 | NR |
| 8G | Comp | 4G | >0.15-<0.45 | NR |
| 8H | CEO | 4H | >0.15-<0.45 | NR |
| 8I | Comp | 4I | >0.15-<0.45 | NR |
| 8J | CEO | 4J | >0.15-<0.45 | NR |
| 8K | Comp | 4K | >0.15-<0.45 | NR |
| 8L | CEO | 4L | >0.15-<0.45 | NR |
| 8M | CEO | 4M | >0.15-<0.45 | NR |
| 8N | Comp | 4N | >0.15-<0.45 | NR |
| 8O | CEO | 4O | >0.15-<0.45 | NR |
| 8P | Comp | 4P | >0.15-<0.45 | NR |
| 8Q | CEO | 4Q | >0.15-<0.45 | NR |
| 8R | Comp | 4R | >0.15-<0.45 | NR |
| 8S | CEO | 4S | >0.15-<0.45 | NR |
| 8T | CEO | 4T | >0.15-<0.45 | NR |
| 8U | CEO | 4U | >0.15-<0.45 | NR |
| 9A | Comp | 5A | >0.15-<0.45 | NR |
| 9B | CEO | 5B | >0.15-<0.45 | NR |
| 9C | Comp | 5C | >0.15-<0.45 | NR |
| 9D | CEO | 5D | >0.15-<0.45 | NR |
| 9E | Comp | 5E | >0.15-<0.45 | NR |
| 9F | CEO | 5F | >0.15-<0.45 | NR |
| 9G | Comp | 5G | >0.15-<0.45 | NR |
| 9H | CEO | 5H | >0.15-<0.45 | NR |
| 9I | Comp | 5I | >0.15-<0.45 | NR |
| 9J | CEO | 5J | >0.15-<0.45 | NR |
| 9K | Comp | 5K | >0.15-<0.45 | NR |
| 9L | CEO | 5L | >0.15-<0.45 | NR |
| 9M | CEO | 5M | >0.15-<0.45 | NR |
| 9N | Comp | 5N | >0.15-<0.45 | NR |
| 9O | CEO | 5O | >0.15-<0.45 | NR |
| 9P | Comp | 5P | >0.15-<0.45 | NR |
| 9Q | CEO | 5Q | >0.15-<0.45 | NR |
| 9R | Comp | 5R | >0.15-<0.45 | NR |
| 9S | CEO | 5S | >0.15-<0.45 | NR |
| 9T | CEO | 5T | >0.15-<0.45 | NR |
| 9U | CEO | 5U | >0.15-<0.45 | NR |
| 10A | Comp | 6A | >0.15-<0.45 | NR |
| 10B | CEO | 6B | >0.15-<0.45 | NR |
| 10C | Comp | 6C | >0.15-<0.45 | NR |
| 10D | CEO | 6D | >0.15-<0.45 | NR |
| 10E | Comp | 6E | >0.15-<0.45 | NR |
| 10F | CEO | 6F | >0.15-<0.45 | NR |
| 10G | Comp | 6G | >0.15-<0.45 | NR |
| 10H | CEO | 6H | >0.15-<0.45 | NR |
| 10I | Comp | 6I | >0.15-<0.45 | NR |
| 10J | CEO | 6J | >0.15-<0.45 | NR |
| 10K | Comp | 6K | >0.15-<0.45 | NP |
| 10L | CEO | 6L | >0.15-<0.45 | NR |
| 10M | CEO | 6M | >0.15-<0.45 | NR |
| 10N | Comp | 6N | >0.15-<0.45 | NR |
| 10O | CEO | 6O | >0.15-<0.45 | NP |
| 10P | Comp | 6P | >0.15-<0.45 | NR |
| 10Q | CEO | 6Q | >0.15-<0.45 | NR |
| 10R | Comp | 6R | >0.15-<0.45 | NR |
| 10S | CEO | 6S | >0.15-<0.45 | NR |
| 10T | CEO | 6T | >0.15-<0.45 | NR |
| 10U | CEO | 6U | >0.15-<0.45 | NR |
| 11A | Comp | 7A | >0.15-<0.45 | NR |
| 11B | CEO | 7B | >0.15-<0.45 | NR |
| 11C | Comp | 7C | >0.15-<0.45 | NF |
| 11D | CEO | 7D | >0.15-<0.45 | NR |
| 11E | Comp | 7E | >0.15-<0.45 | NF |
| 11F | CEO | 7F | >0.15-<0.45 | NR |
| 11G | Comp | 7G | >0.15-<0.45 | NF |
| 11H | CEO | 7H | >0.15-<0.45 | NR |
| 11I | Comp | 7I | >0.15-<0.45 | NR |
| 11J | CEO | 7J | >0.15-<0.45 | NR |
| 11K | Comp | 7K | >0.15-<0.45 | NR |
| 11L | CEO | 7L | >0.15-<0.45 | NR |
| 11M | CEO | 7M | >0.15-<0.45 | NR |
| 11N | Comp | 7N | >0.15-<0.45 | NR |
| 11O | CEO | 7O | >0.15-<0.45 | NR |
| 11P | Comp | 7P | >0.15-<0.45 | NR |
| 11Q | CEO | 7Q | >0.15-<0.45 | NR |
| 11R | Comp | 7R | >0.15-<0.45 | NR |
| 11S | CEO | 7S | >0.15-<0.45 | NR |
| 11T | CEO | 7T | >0.15-<0.45 | NR |
| 11U | CEO | 7U | >0.15-<0.45 | NR |
| 12A | Comp | 8A | >0.15-<0.45 | NR |
| 12B | CEO | 8B | >0.15-<0.45 | NR |
| 12C | Comp | 8C | >0.15-<0.45 | NR |
| 12D | CEO | 8D | >0.15-<0.45 | NR |
| 12E | Comp | 8E | >0.15-<0.45 | NR |
| 12F | CEO | 8F | >0.15-<0.45 | NR |
| 12G | Comp | 8G | >0.15-<0.45 | NR |
| 12H | CEO | 8H | >0.15-<0.45 | NR |
| 12I | Comp | 8I | >0.15-<0.45 | NR |
| 12J | CEO | 8J | >0.15-<0.45 | NR |
| 12K | Comp | 8K | >0.15-<0.45 | NR |
| 12L | CEO | 8L | >0.15-<0.45 | NR |
| 12M | CEO | 8M | >0.15-<0.45 | NR |
| 12N | Comp | 8N | >0.15-<0.45 | NR |
| 12O | CEO | 8O | >0.15-<0.45 | NR |
| 12P | Comp | 8P | >0.15-<0.45 | NR |
| 12Q | CEO | 8Q | >0.15-<0.45 | NR |
| 12R | Comp | 8R | >0.15-<0.45 | NR |
| 12S | CEO | 8S | >0.15-<0.45 | NR |
| 12T | CEO | 8T | >0.15-<0.45 | NR |
| 12U | CEO | 8U | >0.15-<0.45 | NR |
| 13A | Comp | 9A | >0.15-<0.45 | NR |
| 13B | CEO | 9B | >0.15-<0.45 | NR |
| 13C | Comp | 9C | >0.15-<0.45 | NR |
| 13D | CEO | 9D | >0.15-<0.45 | NR |
| 13E | Comp | 9E | >0.15-<0.45 | NR |
| 13F | CEO | 9F | >0.15-<0.45 | NR |
| 13G | Comp | 9G | >0.15-<0.45 | NR |
| 13H | CEO | 9H | >0.15-<0.45 | NR |
| 13I | Comp | 9I | >0.15-<0.45 | NR |
| 13J | CEO | 9J | >0.15-<0.45 | NR |
| 13K | Comp | 9K | >0.15-<0.45 | NR |
| 13L | CEO | 9L | >0.15-<0.45 | NR |
| 13M | CEO | 9M | >0.15-<0.45 | NR |
| 13N | Comp | 9N | >0.15-<0.45 | NR |
| 13O | CEO | 9O | >0.15-<0.45 | NR |
| 13P | Comp | 9P | >0.15-<0.45 | NR |
| 13Q | CEO | 9Q | >0.15-<0.45 | NR |
| 13R | Comp | 9R | >0.15-<0.45 | NR |
| 13S | CEO | 9S | >0.15-<0.45 | NR |
| 13T | CEO | 9T | >0.15-<0.45 | NR |
| 13U | CEO | 9U | >0.15-<0.45 | NR |
| 14A | Comp | 4A | >0.15-<0.35 | NR |
| 14B | CEO | 4B | >0.15-<0.35 | NR |
| 14C | Comp | 4C | >0.15-<0.35 | NR |
| 14D | CEO | 4D | >0.15-<0.35 | NR |
| 14E | Comp | 4E | >0.15-<0.35 | NR |
| 14F | CEO | 4F | >0.15-<0.35 | NR |
| 14G | Comp | 4G | >0.15-<0.35 | NR |
| 14H | CEO | 4H | >0.15-<0.35 | NR |
| 14I | Comp | 4I | >0.15-<0.35 | NR |
| 14J | CEO | 4J | >0.15-<0.35 | NR |
| 14K | Comp | 4K | >0.15-<0.35 | NR |
| 14L | CEO | 4L | >0.15-<0.35 | NR |
| 14M | CEO | 4M | >0.15-<0.35 | NR |
| 14N | Comp | 4N | >0.15-<0.35 | NR |
| 14O | CEO | 4O | >0.15-<0.35 | NR |
| 14P | Comp | 4P | >0.15-<0.35 | NR |
| 14Q | CEO | 4Q | >0.15-<0.35 | NR |
| 14R | Comp | 4R | >0.15-<0.35 | NR |
| 14S | CEO | 4S | >0.15-<0.35 | NR |
| 14T | CEO | 4T | >0.15-<0.35 | NR |
| 14U | CEO | 4U | >0.15-<0.35 | NR |
| 15A | Comp | 5A | >0.15-<0.35 | NR |
| 15B | CEO | 5B | >0.15-<0.35 | NR |
| 15C | Comp | 5C | >0.15-<0.35 | NR |
| 15D | CEO | 5D | >0.15-<0.35 | NR |
| 15E | Comp | 5E | >0.15-<0.35 | NR |
| 15F | CEO | 5F | >0.15-<0.35 | NR |
| 15G | Comp | 5G | >0.15-<0.35 | NR |
| 15H | CEO | 5H | >0.15-<0.35 | NR |
| 15I | Comp | 5I | >0.15-<0.35 | NR |
| 15J | CEO | 5J | >0.15-<0.35 | NR |
| 15K | Comp | 5K | >0.15-<0.35 | NR |
| 15L | CEO | 5L | >0.15-<0.35 | NR |
| 15M | CEO | 5M | >0.15-<0.35 | NR |
| 15N | Comp | 5N | >0.15-<0.35 | NR |
| 15O | CEO | 5O | >0.15-<0.35 | NR |
| 15P | Comp | 5P | >0.15-<0.35 | NR |
| 15Q | CEO | 5Q | >0.15-<0.35 | NR |
| 15R | Comp | 5R | >0.15-<0.35 | NR |
| 15S | CEO | 5S | >0.15-<0.35 | NR |
| 15T | CEO | 5T | >0.15-<0.35 | NR |
| 15U | CEO | 5U | >0.15-<0.35 | NR |
| 16A | Comp | 6A | >0.15-<0.35 | NR |
| 16B | CEO | 6B | >0.15-<0.35 | NR |
| 16C | Comp | 6C | >0.15-<0.35 | NR |
| 16D | CEO | 6D | >0.15-<0.35 | NR |
| 16E | Comp | 6E | >0.15-<0.35 | NR |
| 16F | CEO | 6F | >0.15-<0.35 | NR |
| 16G | Comp | 6G | >0.15-<0.35 | NR |
| 16H | CEO | 6H | >0.15-<0.35 | NR |
| 16I | Comp | 6I | >0.15-<0.35 | NR |
| 16J | CEO | 6J | >0.15-<0.35 | NR |
| 16K | Comp | 6K | >0.15-<0.35 | NR |
| 16L | CEO | 6L | >0.15-<0.35 | NR |
| 16M | CEO | 6M | >0.15-<0.35 | NR |
| 16N | Comp | 6N | >0.15-<0.35 | NR |
| 16O | CEO | 6O | >0.15-<0.35 | NR |
| 16P | Comp | 6P | >0.15-<0.35 | NR |
| 16Q | CEO | 6Q | >0.15-<0.35 | NR |
| 16R | Comp | 6R | >0.15-<0.35 | NR |
| 16S | CEO | 6S | >0.15-<0.35 | NR |
| 16T | CEO | 6T | >0.15-<0.35 | NR |
| 16U | CEO | 6U | >0.15-<0.35 | NR |
| 17A | Comp | 7A | >0.15-<0.35 | NR |
| 17B | CEO | 7B | >0.15-<0.35 | NR |
| 17C | Comp | 7C | >0.15-<0.35 | NR |
| 17D | CEO | 7D | >0.15-<0.35 | NR |
| 17E | Comp | 7E | >0.15-<0.35 | NR |
| 17F | CEO | 7F | >0.15-<0.35 | NR |
| 17G | Comp | 7G | >0.15-<0.35 | NR |
| 17H | CEO | 7H | >0.15-<0.35 | NR |
| 17I | Comp | 7I | >0.15-<0.35 | NR |
| 17J | CEO | 7J | >0.15-<0.35 | NR |
| 17K | Comp | 7K | >0.15-<0.35 | NR |
| 17L | CEO | 7L | >0.15-<0.35 | NR |
| 17M | CEO | 7M | >0.15-<0.35 | NR |
| 17N | Comp | 7N | >0.15-<0.35 | NR |
| 17O | CEO | 7O | >0.15-<0.35 | NP |
| 17P | Comp | 7P | >0.15-<0.35 | NR |
| 17Q | CEO | 7Q | >0.15-<0.35 | NR |
| 17R | Comp | 7R | >0.15-<0.35 | NR |
| 17S | CEO | 7S | >0.15-<0.35 | NF |
| 17T | CEO | 7T | >0.15-<0.35 | NR |
| 17U | CEO | 7U | >0.15-<0.35 | NP |
| 18A | Comp | 8A | >0.15-<0.35 | NR |
| 18B | CEO | 8B | >0.15-<0.35 | NR |
| 18C | Comp | 8C | >0.15-<0.35 | NR |
| 18D | CEO | 8D | >0.15-<0.35 | NR |
| 18E | Comp | 8E | >0.15-<0.35 | NR |
| 18F | CEO | 8F | >0.15-<0.35 | NR |
| 18G | Comp | 8G | >0.15-<0.35 | NR |
| 18H | CEO | 8H | >0.15-<0.35 | NR |
| 18I | Comp | 8I | >0.15-<0.35 | NR |
| 18J | CEO | 8J | >0.15-<0.35 | NR |
| 18K | Comp | 8K | >0.15-<0.35 | NR |
| 18L | CEO | 8L | >0.15-<0.35 | NR |
| 18M | CEO | 8M | >0.15-<0.35 | NR |
| 18N | Comp | 8N | >0.15-<0.35 | NR |
| 18O | CEO | 8O | >0.15-<0.35 | NR |
| 18P | Comp | 8P | >0.15-<0.35 | NR |
| 18Q | CEO | 8Q | >0.15-<0.35 | NR |
| 18R | Comp | 8R | >0.15-<0.35 | NR |
| 18S | CEO | 8S | >0.15-<0.35 | NR |
| 18T | CEO | 8T | >0.15-<0.35 | NF |
| 18U | CEO | 8U | >0.15-<0.35 | NR |
| 19A | Comp | 9A | >0.15-<0.35 | NR |
| 19B | CEO | 9B | >0.15-<0.35 | NR |
| 19C | Comp | 9C | >0.15-<0.35 | NR |
| 19D | CEO | 9D | >0.15-<0.35 | NR |
| 19E | Comp | 9E | >0.15-<0.35 | NR |
| 19F | CEO | 9F | >0.15-<0.35 | NR |
| 19G | Comp | 9G | >0.15-<0.35 | NR |
| 19H | CEO | 9H | >0.15-<0.35 | NR |
| 19I | Comp | 9I | >0.15-<0.35 | NR |
| 19J | CEO | 9J | >0.15-<0.35 | NR |
| 19K | Comp | 9K | >0.15-<0.35 | NR |
| 19L | CEO | 9L | >0.15-<0.35 | NR |
| 19M | CEO | 9M | >0.15-<0.35 | NR |
| 19N | Comp | 9N | >0.15-<0.35 | NR |
| 19O | CEO | 9O | >0.15-<0.35 | NR |
| 19P | Comp | 9P | >0.15-<0.35 | NR |
| 19Q | CEO | 9Q | >0.15-<0.35 | NR |
| 19R | Comp | 9R | >0.15-<0.35 | NR |
| 19S | CEO | 9S | >0.15-<0.35 | NR |
| 19T | CEO | 9T | >0.15-<0.35 | NR |
| 19U | CEO | 9U | >0.15-<0.35 | NR |
| 20A | Comp | 4A | <0.5 | >40% |
| 20B | CEO | 4B | <0.5 | >40% |
| 20C | Comp | 4C | <0.5 | >40% |
| 20D | CEO | 4D | <0.5 | >40% |
| 20E | Comp | 4E | <0.5 | >40% |
| 20F | CEO | 4F | <0.5 | >40% |
| 20G | Comp | 4G | <0.5 | >40% |
| 20H | CEO | 4H | <0.5 | >40% |
| 20I | Comp | 4I | <0.5 | >40% |
| 20J | CEO | 4J | <0.5 | >40% |
| 20K | Comp | 4K | <0.5 | >40% |
| 20L | CEO | 4L | <0.5 | >40% |
| 20M | CEO | 4M | <0.5 | >40% |
| 20N | Comp | 4N | <0.5 | >40% |
| 20O | CEO | 4O | <0.5 | >40% |
| 20P | Comp | 4P | <0.5 | >40% |
| 20Q | CEO | 4G | <0.5 | >40% |
| 20R | Comp | 4R | <0.5 | >40% |
| 20S | CEO | 4S | <0.5 | >40% |
| 20T | CEO | 4T | <0.5 | >40% |
| 20U | CEO | 4U | <0.5 | >40% |
| 21A | Comp | 5A | <0.5 | >40% |
| 21B | CEO | 5B | <0.5 | >40% |
| 21C | Comp | 5C | <0.5 | >40% |
| 21D | CEO | 5D | <0.5 | >40% |
| 21E | Comp | 5E | <0.5 | >40% |
| 21F | CEO | 5F | <0.5 | >40% |
| 21G | Comp | 5G | <0.5 | >40% |
| 21H | CEO | 5H | <0.5 | >40% |
| 21I | Comp | 5I | <0.5 | >40% |
| 21J | CEO | 5J | <0.5 | >40% |
| 21K | Comp | 5K | <0.5 | >40% |
| 21L | CEO | 5L | <0.5 | >40% |
| 21M | CEO | 5M | <0.5 | >40% |
| 21N | Comp | 5N | <0.5 | >40% |
| 21O | CEO | 5O | <0.5 | >40% |
| 21P | Comp | 5P | <0.5 | >40% |
| 21Q | CEO | 5G | <0.5 | >40% |
| 21R | Comp | 5R | <0.5 | >40% |
| 21S | CEO | 5S | <0.5 | >40% |
| 21T | CEO | 5T | <0.5 | >40% |
| 21U | CEO | 5U | <0.5 | >40% |
| 22A | Comp | 6A | <0.5 | >40% |
| 22B | CEO | 6B | <0.5 | >40% |
| 22C | Comp | 6C | <0.5 | >40% |
| 22D | CEO | 6D | <0.5 | >40% |
| 22E | Comp | 6E | <0.5 | >40% |
| 22F | CEO | 6F | <0.5 | >40% |
| 22G | Comp | 6G | <0.5 | >40% |
| 22H | CEO | 6H | <0.5 | >40% |
| 22I | Comp | 6I | <0.5 | >40% |
| 22J | CEO | 6J | <0.5 | >40% |
| 22K | Comp | 6K | <0.5 | >40% |
| 22L | CEO | 6L | <0.5 | >40% |
| 22M | CEO | 6M | <0.5 | >40% |
| 22N | Comp | 6N | <0.5 | >40% |
| 22O | CEO | 6O | <0.5 | >40% |
| 22P | Comp | 6P | <0.5 | >40% |
| 22Q | CEO | 6Q | <0.5 | >40% |
| 22R | Comp | 6R | <0.5 | >40% |
| 22S | CEO | 6S | <0.5 | >40% |
| 22T | CEO | 6T | <0.5 | >40% |
| 22U | CEO | 6U | <0.5 | >40% |
| 23A | Comp | 7A | <0.5 | >40% |
| 23B | CEO | 7B | <0.5 | >40% |
| 23C | Comp | 7C | <0.5 | >40% |
| 23D | CEO | 7D | <0.5 | >40% |
| 23E | Comp | 7E | <0.5 | >40% |
| 23F | CEO | 7F | <0.5 | >40% |
| 23G | Comp | 7G | <0.5 | >40% |
| 23H | CEO | 7H | <0.5 | >40% |
| 23I | Comp | 7I | <0.5 | >40% |
| 23J | CEO | 7J | <0.5 | >40% |
| 23K | Comp | 7K | <0.5 | >40% |
| 23L | CEO | 7L | <0.5 | >40% |
| 23M | CEO | 7N | <0.5 | >40% |
| 23N | Comp | 7N | <0.5 | >40% |
| 23O | CEO | 7O | <0.5 | >40% |
| 23P | Comp | 7P | <0.5 | >40% |
| 23Q | CEO | 7Q | <0.5 | >40% |
| 23R | Comp | 7R | <0.5 | >40% |
| 23S | CEO | 7S | <0.5 | >40% |
| 23T | CEO | 7T | <0.5 | >40% |
| 23U | CEO | 7U | <0.5 | >40% |
| 24A | Comp | 8A | <0.5 | >40% |
| 24B | CEO | 8B | <0.5 | >40% |
| 24C | Comp | 8C | <0.5 | >40% |
| 24D | CEO | 8D | <0.5 | >40% |
| 24E | Comp | 8E | <0.5 | >40% |
| 24F | CEO | 8F | <0.5 | >40% |
| 24G | Comp | 8G | <0.5 | >40% |
| 24H | CEO | 8H | <0.5 | >40% |
| 24I | Comp | 8I | <0.5 | >40% |
| 24J | CEO | 8J | <0.5 | >40% |
| 24K | Comp | 8K | <0.5 | >40% |
| 24L | CEO | 8L | <0.5 | >40% |
| 24M | CEO | 8M | <0.5 | >40% |
| 24N | Comp | 8N | <0.5 | >40% |
| 24O | CEO | 8O | <0.5 | >40% |
| 24P | Comp | 8P | <0.5 | >40% |
| 24Q | CEO | 8Q | <0.5 | >40% |
| 24R | Comp | 8R | <0.5 | >40% |
| 24S | CEO | 8S | <0.5 | >40% |
| 24T | CEO | 8T | <0.5 | >40% |
| 24U | CEO | 8U | <0.5 | >40% |
| 25A | Comp | 9A | <0.5 | >40% |
| 25B | CEO | 9B | <0.5 | >40% |
| 25C | Comp | 9C | <0.5 | >40% |
| 25D | CEO | 9D | <0.5 | >40% |
| 25E | Comp | 9E | <0.5 | >40% |
| 25F | CEO | 9F | <0.5 | >40% |
| 25G | Comp | 9G | <0.5 | >40% |
| 25H | CEO | 9H | <0.5 | >40% |
| 25I | Comp | 9I | <0.5 | >40% |
| 25J | CEO | 9J | <0.5 | >40% |
| 25K | Comp | 9K | <0.5 | >40% |
| 25L | CEO | 9L | <0.5 | >40% |
| 25M | CEO | 9M | <0.5 | >40% |
| 25N | Comp | 9N | <0.5 | >40% |
| 25O | CEO | 9O | <0.5 | >40% |
| 25P | Comp | 9P | <0.5 | >40% |
| 25Q | CEO | 9Q | <0.5 | >40% |
| 25R | Comp | 9R | <0.5 | >40% |
| 25S | CEO | 9S | <0.5 | >40% |
| 25T | CEO | 9T | <0.5 | >40% |
| 25U | CEO | 9U | <0.5 | >40% |
| 26A | Comp | 4A | >0.15-<0.45 | >45% |
| 26B | CEO | 4B | >0.15-<0.45 | >45% |
| 26C | Comp | 4C | >0.15-<0.45 | >45% |
| 26D | CEO | 4D | >0.15-<0.45 | >45% |
| 26E | Comp | 4E | >0.15-<0.45 | >45% |
| 26F | CEO | 4F | >0.15-<0.45 | >45% |
| 26G | Comp | 4G | >0.15-<0.45 | >45% |
| 26H | CEO | 4H | >0.15-<0.45 | >45% |
| 26I | Comp | 4I | >0.15-<0.45 | >45% |
| 26J | CEO | 4J | >0.15-<0.45 | >45% |
| 26K | Comp | 4K | >0.15-<0.45 | >45% |
| 26L | CEO | 4L | >0.15-<0.45 | >45% |
| 26M | CEO | 4M | >0.15-<0.45 | >45% |
| 26N | Comp | 4N | >0.15-<0.45 | >45% |
| 26O | CEO | 4O | >0.15-<0.45 | >45% |
| 26P | Comp | 4P | >0.15-<0.45 | >45% |
| 26Q | CEO | 4Q | >0.15-<0.45 | >45% |
| 26R | Comp | 4R | >0.15-<0.45 | >45% |
| 26S | CEO | 4S | >0.15-<0.45 | >45% |
| 26T | CEO | 4T | >0.15-<0.45 | >45% |
| 26U | CEO | 4U | >0.15-<0.45 | >45% |
| 27A | Comp | 5A | >0.15-<0.45 | >45% |
| 27B | CEO | 5B | >0.15-<0.45 | >45% |
| 27C | Comp | 5C | >0.15-<0.45 | >45% |
| 27D | CEO | 5D | >0.15-<0.45 | >45% |
| 27E | Comp | 5E | >0.15-<0.45 | >45% |
| 27F | CEO | 5F | >0.15-<0.45 | >45% |
| 27G | Comp | 5G | >0.15-<0.45 | >45% |
| 27H | CEO | 5H | >0.15-<0.45 | >45% |
| 27I | Comp | 5I | >0.15-<0.45 | >45% |
| 27J | CEO | 5J | >0.15-<0.45 | >45% |
| 27K | Comp | 5K | >0.15-<0.45 | >45% |
| 27L | CEO | 5L | >0.15-<0.45 | >45% |
| 27M | CEO | 5M | >0.15-<0.45 | >45% |
| 27N | Comp | 5N | >0.15-<0.45 | >45% |
| 27O | CEO | 5O | >0.15-<0.45 | >45% |
| 27P | Comp | 5P | >0.15-<0.45 | >45% |
| 27Q | CEO | 5Q | >0.15-<0.45 | >45% |
| 27R | Comp | 5R | >0.15-<0.45 | >45% |
| 27S | CEO | 5S | >0.15-<0.45 | >45% |
| 27T | CEO | 5T | >0.15-<0.45 | >45% |
| 27U | CEO | 5U | >0.15-<0.45 | >45% |
| 28A | Comp | 6A | >0.15-<0.45 | >45% |
| 28B | CEO | 6B | >0.15-<0.45 | >45% |
| 28C | Comp | 6C | >0.15-<0.45 | >45% |
| 28D | CEO | 6D | >0.15-<0.45 | >45% |
| 28E | Comp | 6E | >0.15-<0.45 | >45% |
| 28F | CEO | 6F | >0.15-<0.45 | >45% |
| 28G | Comp | 6G | >0.15-<0.45 | >45% |
| 28H | CEO | 6H | >0.15-<0.45 | >45% |
| 28I | Comp | 6I | >0.15-<0.45 | >45% |
| 28J | CEO | 6J | >0.15-<0.45 | >45% |
| 28K | Comp | 6K | >0.15-<0.45 | >45% |
| 28L | CEO | 6L | >0.15-<0.45 | >45% |
| 28M | CEO | 6M | >0.15-<0.45 | >45% |
| 28N | Comp | 6N | >0.15-<0.45 | >45% |
| 28O | CEO | 6O | >0.15-<0.45 | >45% |
| 28P | Comp | 6P | >0.15-<0.45 | >45% |
| 28Q | CEO | 6Q | >0.15-<0.45 | >45% |
| 28R | Comp | 6R | >0.15-<0.45 | >45% |
| 28S | CEO | 6S | >0.15-<0.45 | >45% |
| 28T | CEO | 6T | >0.15-<0.45 | >45% |
| 28U | CEO | 6U | >0.15-<0.45 | >45% |
| 29A | Comp | 7A | >0.15-<0.45 | >45% |
| 29B | CEO | 7B | >0.15-<0.45 | >45% |
| 29C | Comp | 7C | >0.15-<0.45 | >45% |
| 29D | CEO | 7D | >0.15-<0.45 | >45% |
| 29E | Comp | 7E | >0.15-<0.45 | >45% |
| 29F | CEO | 7F | >0.15-<0.45 | >45% |
| 29G | Comp | 7G | >0.15-<0.45 | >45% |
| 29H | CEO | 7H | >0.15-<0.45 | >45% |
| 29I | Comp | 7I | >0.15-<0.45 | >45% |
| 29J | CEO | 7J | >0.15-<0.45 | >45% |
| 29K | Comp | 7K | >0.15-<0.45 | >45% |
| 29L | CEO | 7L | >0.15-<0.45 | >45% |
| 29M | CEO | 7M | >0.15-<0.45 | >45% |
| 29N | Comp | 7N | >0.15-<0.45 | >45% |
| 29O | CEO | 7O | >0.15-<0.45 | >45% |
| 29P | Comp | 7P | >0.15-<0.45 | >45% |
| 29Q | CEO | 7Q | >0.15-<0.45 | >45% |
| 29R | Comp | 7R | >0.15-<0.45 | >45% |
| 29S | CEO | 79 | >0.15-<0.45 | >45% |
| 29T | CEO | 7T | >0.15-<0.45 | >45% |
| 29U | CEO | 7U | >0.15-<0.45 | >45% |
| 30A | Comp | 8A | >0.15-<0.45 | >45% |
| 30B | CEO | 8B | >0.15-<0.45 | >45% |
| 30C | Comp | 8C | >0.15-<0.45 | >45% |
| 30D | CEO | 8D | >0.15-<0.45 | >45% |
| 30E | Comp | 8E | >0.15-<0.45 | >45% |
| 30F | CEO | 8F | >0.15-<0.45 | >45% |
| 30G | Comp | 8G | >0.15-<0.45 | >45% |
| 30H | CEO | 8H | >0.15-<0.45 | >45% |
| 30I | Comp | 8I | >0.15-<0.45 | >45% |
| 30J | CEO | 8J | >0.15-<0.45 | >45% |
| 30K | Comp | 8K | >0.15-<0.45 | >45% |
| 30L | CEO | 8L | >0.15-<0.45 | >45% |
| 30M | CEO | 8M | >0.15-<0.45 | >45% |
| 30N | Comp | 8N | >0.15-<0.45 | >45% |
| 30O | CEO | 8O | >0.15-<0.45 | >45% |
| 30P | Comp | 8P | >0.15-<0.45 | >45% |
| 30Q | CEO | 8Q | >0.15-<0.45 | >45% |
| 30R | Comp | 8R | >0.15-<0.45 | >45% |
| 30S | CEO | 8S | >0.15-<0.45 | >45% |
| 30T | CEO | 8T | >0.15-<0.45 | >45% |
| 30U | CEO | 8U | >0.15-<0.45 | >45% |
| 31A | Comp | 9A | >0.15-<0.45 | >45% |
| 31B | CEO | 9B | >0.15-<0.45 | >45% |
| 31C | Comp | 9C | >0.15-<0.45 | >45% |
| 31D | CEO | 9D | >0.15-<0.45 | >45% |
| 31E | Comp | 9E | >0.15-<0.45 | >45% |
| 31F | CEO | 9F | >0.15-<0.45 | >45% |
| 31G | Comp | 9G | >0.15-<0.45 | >45% |
| 31H | CEO | 9H | >0.15-<0.45 | >45% |
| 31I | Comp | 9I | >0.15-<0.45 | >45% |
| 31J | CEO | 9J | >0.15-<0.45 | >45% |
| 31K | Comp | 9K | >0.15-<0.45 | >45% |
| 31L | CEO | 9L | >0.15-<0.45 | >45% |
| 31M | CEO | 9M | >0.15-<0.45 | >45% |
| 31N | Comp | 9N | >0.15-<0.45 | >45% |
| 31O | CEO | 9O | >0.15-<0.45 | >45% |
| 31P | Comp | 9P | >0.15-<0.45 | >45% |
| 31Q | CEO | 9Q | >0.15-<0.45 | >45% |
| 31R | Comp | 9R | >0.15-<0.45 | >45% |
| 31S | CEO | 9S | >0.15-<0.45 | >45% |
| 31T | CEO | 9T | >0.15-<0.45 | >45% |
| 31U | CEO | 9U | >0.15-<0.45 | >45% |

The present invention thus provides pressurized metered dose inhalers (MDIs), including each of MDI1A through MDI1D, for the treatment of asthma and other chronic obstructive pulmonary diseases or other diseases as are known to be treatable by those skilled in the art by ipratropium by delivery thereof to the lungs of the patient.

The present invention thus includes methods for delivering of pharmaceutical compositions, including Pharmaceutical Delivery Methods 1A through 1D, for purpose of treating ailments, diseases and similar health related problems of an organism (such as a human or animal) comprising applying a composition of the present invention containing ipratropium, including any and all pharmaceutically effective salts thereof, to the organism in need of treatment.

The MDI metering valve size, that is, the size of the metering chamber, can vary within the scope hereof, but may be between 10 microliters (μL or mcl) and 100 microliters, or from about 25 to about −90, or from about 40 microliters to about 80 microliters, or from about 50 to about 70, or from about 60 to about 65 microliters.

In certain embodiments, pMDIs of the present invention, including each of MDI1A through MDI1D and methods of the present disclosure use a pMDI of the present invention, deliver ipratropium in an amount of from about 10 to about 100 milligram per actuation (mg/actuation), or about 10 to about 50 milligram per actuation (mg/actuation), or about 20 to about 30 milligram per actuation (mg/actuation).

The present invention includes methods of forming pharmaceutical compositions, including each of Pharmaceutical Compositions 1-3 and PC 4-9 and forming an aerosol comprising ipratropium bromide by forcing said pharmaceutical composition through an orifice having a diameter of less than 0.5 mm.

The present invention includes methods of forming pharmaceutical compositions, including each of Pharmaceutical Compositions 1-3 and PC 4-9 and forming an aerosol comprising ipratropium bromide by forcing said pharmaceutical composition through an orifice having a diameter of from about 0.2 mm to about 0.45. The present invention includes methods of forming pharmaceutical compositions, including each of Pharmaceutical Compositions 1-3 and PC 4-9 and forming an aerosol comprising ipratropium bromide by forcing said pharmaceutical composition through an orifice having a diameter of from about 0.2 mm to about 0.4 mm.

The present invention includes methods of forming pharmaceutical compositions, including each of Pharmaceutical Compositions 1-3 and PC 4-9 and forming an aerosol comprising ipratropium bromide by forcing said pharmaceutical composition through an orifice having a diameter of from about 0.2 mm to about 0.35 mm.

The present invention includes methods of forming pharmaceutical compositions, including each of Pharmaceutical Compositions 1-3 and PC 4-9 and forming an aerosol comprising ipratropium bromide by forcing said pharmaceutical composition through an orifice having a diameter of from about 0.2 mm to about 0.30 mm.

### EXAMPLES

### Comparative Example 1—Formulation of Ipratroplum, HFC-134a, Ethanol and Citric Acid

Ipratropium bromide (IB) as the API for the example was procured from MilliporeSigma (St Louis, MO, USA). Propellant grade HFC-134a was supplied by Honeywell (Morris Plains, NJ, USA). USP grade absolute ethanol was purchased from Fisher Scientific (Waltham, MA, USA). An IB solution in ethanol, water and citric acid was weighed directly into a suitable canister. A quantitative amount of ethanol in the final solution (about 15 wt % ethanol and about 0.5% water and 0.011 citric acid in the final formulation) was dispensed into the canister, and the canisters were fitted with metering valves as identified below in Comparative Example 2. Sealed canisters were then pressure-filled with HFC-134a propellant. Canisters were agitated to ensure adequate mixing and then left to equilibrate under 2-week quarantine. The resulting formulation consisted of ipratropium bromide (in solution), water, HFC-134a, ethanol and citric acid (as a stabilizer) in the concentrations identified in Table CEx1 below:

TABLE CEX1

| | FORMULATION |
|---|---|
| Components | C1, wt % |
| HFC-134a | 84.455 |
| Ethanol | 15 |
| Ipratropium bromide | 00.034 |
| Citric acid | 00.011 |
| Water | 00.500 |
| Total | 100.00 |

### Comparative Examples 2A and 2B—Particle Size Distribution of Formulation C1 Using 0.5 mm and 0.22 Orifice

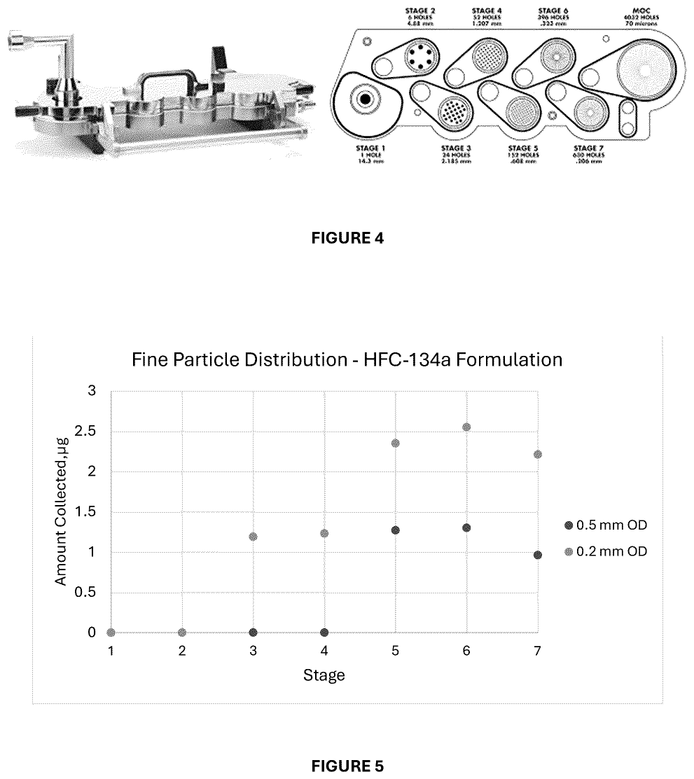

The pMDI canisters of Comparative Example 1 were fitted onto 63 μl valves, and the valved cannisters were then mounted in a first series of actuators having a 0.50 mm outside diameter orifice and in second series of actuators a 0.22 mm outside diameter orifice, to produce a pMDI generally as described above in connection FIGS. 1-3. Except for the different actuator orifice sizes, the pMDIs were identical. Aerodynamic particle size assessments of the pMDIs were conducted using a Next Generation Impactor (NGI) (USP Test Chapter 601) fitted with a USP induction port at the beginning and end of canister-use life from each of two batches. Each determination was obtained by sampling 15 consecutive doses at a sampling flow rate of 28.3 l/minute (1SCFM). For each canister tested, the delivered dose was determined using DUSA methodology (Dose Unit Spray Apparatus) at the beginning, middle, and end of canister-use life. Quantification of IP and FPF within test samples was performed using a HPLC method. The apparatus used in illustrated in FIG. 4, and the particle size cut-off diameter for each stage of the device is provided in the Table CEx2A below:

TABLE CEX2A
CUT-OFF DIAMETERS FOR EACH STAGE

| NGI Stage | Cut-off Diameter (um) |
|---|---|
| Stage 1 | 11.719 |
| Stage 2 | 6.395 |
| Stage 3 | 3.988 |
| Stage 4 | 2.299 |
| Stage 5 | 1.357 |
| Stage 6 | 0.834 |
| Stage 7 | 0.541 |

As is known to those skilled in the art, cascade impaction as per this example is an accepted technique for the measurement of how particle sizes will be distributed in the human body from the pMDI in use since multistage cascade impactors separate samples on the basis of difference in particle inertia. Penetration to the lung is generally considered to occur for particle sizes of less than 5 microns (μm), which is the standard used to define “fine particle dose” (FPD) and the basis for determining the fine particle fraction (FPF), which is the FPD divided by delivered dose (DD), that is, the total amount of the drug delivered from the pMDI actuator per actuation. In this example, therefore, the FPF is determined by totaling the collection in Stages 3-7 and dividing this value by the DD.

The average results of the amount of particles collected as a function of location/stage are reported in Table CEx2B below and illustrated in the data plot for stages 1-7 in FIG. 5.

TABLE CEX2B

Example No./ORIFICE SIZE, mm

| | |
|---|---|
| C2A/0.5 | C2B/0.22 |

| | |
|---|---|
| Location/Stage | Amount, μg |

| | | |
|---|---|---|
| Valve/Actuator | 3.56 | 6.60 |
| Throat | 16.90 | 12.17 |
| Stage 1 | 0.00 | 0.00 |
| Stage 2 | 0.00 | 0.00 |
| Stage 3 | 0.00 | 1.19 |
| Stage 4 | 0.00 | 1.23 |
| Stage 5 | 1.27 | 2.35 |
| Stage 6 | 1.30 | 2.55 |
| Stage 7 | 0.96 | 2.21 |
| Totals, μg | | |
| Leaving Valve | 25.11 | 30.67 |
| Leaving Actuator | 21.55 | 24.07 |
| Collected in Stages 3 | 3.53 | 9.53 |
| through 7 (<5 micron) | | |
| FPF | 16.39% | 39.59% |

As can be seen from the above data and charts presented in the Figures, the fine particle fraction increases as expected as the orifice diameter decreases, that is, from 16.39% to 39.59%. This result is expected since it has been observed that generally the amount of fine particles produced increases as orifice diameter decreases when the propellant is HFC-134a. Although applicants do not necessarily intend to be bound by their theory of operation, applicants generally believe that the relatively high vapor pressure of the HFC-134a propellant causes increased shear as the formulation exits the orifice, which in turn results in increased production of fine particles.

### Example 1A, 1B and 1C—Formulation of Ipratropium, HFO-1234ze(E), Ethanol and Citric Acid

A stable solution formulation was prepared consisting of ipratropium bromide (“IB”), water, HFO-1234ze(E), ethanol and citric acid (as a stabilizer) in accordance with the procedures described in Comparative Example 1 and having the concentrations as reported in Table EX1 under Formulation 1A below. Propellant grade HFO-1234ze(E) was supplied by Honeywell (Morris Plains, NJ, USA). Formulations 1B and 1C are also formulated as reported in Table EX1.

TABLE EX1

| | FORMULATIONS, wt % | | |
|---|---|---|---|
| Components | 1A | 1B | 1C |
| HFO-1234ze(E) | 84.455 | 81.455 | 79.455 |
| Ethanol | 15 | 18 | 20 |
| Ipratropium bromide | 0.034 | 00.034 | 0.034 |
| Citric acid | 0.011 | 00.011 | .011 |
| Water | 0.497 | 00.497 | .497 |
| Total | 100.00 | 100.00 | 100.00 |

Each of these formulations was tested for stability by storing in FEP coated MDI canisters at 40° C. and 75% relative humidity for 12 weeks. At week 12, the ipratropium bromide content and the delivered dose were/are found to exhibit acceptable solution stability.

### Comparative Example 3—Particle Size Distribution of Formulation 1A Using 0.5 mm Orifice

Comparative Example 2B was repeated, except formulation 1A using HFO-1234ze(E) was used to replace HFC-134a on a weight per weight basis as the propellant. The average results of the amount of particle sizes collected as a function of location/stage are reported in Table CEx2.

TABLE CEX2

| | Formulation No./Orifice |
|---|---|
| | Diameter, mm |
| Location/Stage | 1A/0.5 |
| Valve/Actuator | 2.20 |
| Throat | 12.83 |
| Stage 1 | 10.26 |
| Stage 2 | 0.00 |
| Stage 3 | 0.00 |
| Stage 4 | 0.00 |
| Stage 5 | 0.00 |
| Stage 6 | 0.00 |
| Stage 7 | 0.00 |
| Totals, μg | |
| Leaving Valve | 25.29 |
| Leaving Actuator | 23.09 |
| Collected in Stages 3 | 0 |
| through 7 (<5 micron) | |
| FPF | 0% |

As can be seen from the results above, the formulation containing HFO-1234ze(E) as the propellant and using a 0.5 mm orifice size resulted in the production of 0% FPF. Based on this result, it would appear that HFO-1234ze(E) would not be a successful replacement for HFC-134a as the propellant for ipratropium formulations in pMDIs. Although applicants do not necessarily intend to be bound by their theory of operation, this result is not completely unexpected since HFO-1234ze(E) has a lower vapor pressure than the vapor pressure of HFC-134a (e.g., normal boiling point of −16° C. for 1234ze(E) compared to the normal boiling point of −29° C. for HFC-134a), and the lower vapor pressure would generally be expected to result in less shear forces as the formulation exits the orifice and therefore resulting in a lower production fine particles. However, it cannot be said that a result of FPF of zero percent was expected.

### Example 2A—Particle Size Distribution of Formulation Ex1A with 1234Ze(E) Using 0.22 mm Orifice

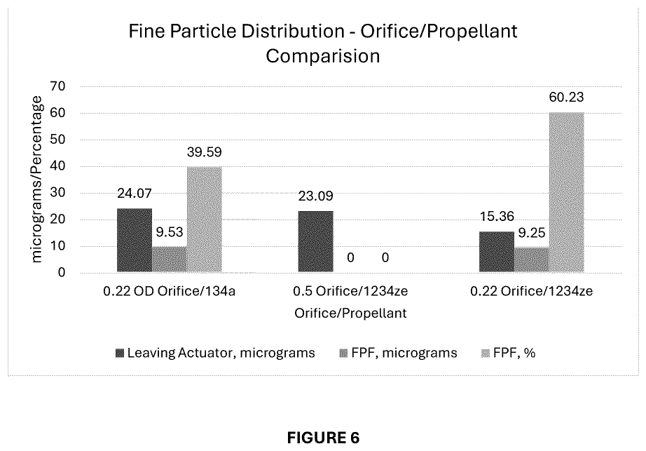

Comparative Example 3 was repeated using the same formulation Ex A containing about 85 wt % HFO-1234ze(E), except that the 0.22 mm orifice of Comparative Example 2A was used. The average results of the amount of particle sizes collected as a function of location/stage collected are reported in Table Ex2A below, together with the results of Comparative Example C2B and Comparative Example C3 for ease of comparison, and illustrated in the data plot for stages 1-7 in FIG. 6.

TABLE EX2A

| | Example No./ORIFICE SIZE, mm | | |
|---|---|---|---|
| | ExC2A/0.22 | ExC3/0.50 | Ex2A/0.22 |
| | Formulation | | |
| Formulation | C1 (91.5% | 1A (91.5% | 1A (91.5% |
| Location/Stage | 134a) | 1234ze(E)) | 1234ze(E)) |
| Valve/Actuator | 6.60 | 2.20 | 7.73 |
| Throat | 12.17 | 12.83 | 4.13 |
| Stage 1 | 0.00 | 10.26 | 0.00 |
| Stage 2 | 0.00 | 0.00 | 0.00 |
| Stage 3 | 1.19 | 0.00 | 0.00 |
| Stage 4 | 1.23 | 0.00 | 1.49 |
| Stage 5 | 2.35 | 0.00 | 2.64 |
| Stage 6 | 2.55 | 0.00 | 2.96 |
| Stage 7 | 2.21 | 0.00 | 2.16 |
| Totals, μg | | | |
| Leaving Valve | 30.67 | 25.29 | 23.09 |
| Leaving Actuator | 24.07 | 23.09 | 15.36 |
| Collected in Stages | 9.53 | 0 | 9.25 |
| 3 through 7 (<5 | | | |
| micron) | | | |
| FPF | 39.59% | 0% | 60.23% |
| FPF Improvement | 1 | Substantial | 1.52 |
| (Relative to R134a | | decline | |
| at 1) | | | |

As can be seen from the results reported in the example, while the total particles leaving the actuator using the 0.22 mm valve was about the same as when using the 0.5 mm valve, the FPF was unexpectedly dramatically increased to a value of just over 60%. Furthermore, this result is unexpectedly not only better than, but nearly double, the FPF value achieved when the higher vapor pressure HFC-134a is used as the propellant with the 0.22 OD orifice valve. One important advantage of this unexpected result is that a proportionally increased number of effective doses can supplied from the pMDI according to the present invention compared to both the use of HFC-134a with the 0.22 orifice diameter valve and using HFO-1234ze(E) using the 0.5 OD valve.

### Example 2B—Particle Size Distribution of Formulation Ex1B with 1234Ze(E) Using 0.22 mm Orifice

Comparative 3 and Example 2A are repeated, except using the formulation 1B as described in Example 1. A similar unexpected improvement in FPF, and its associated advantage, is achieved.

### Example 2C—Particle Size Distribution of Formulation Ex1B with 1234Ze(E) Using 0.22 mm Orifice

Comparative 3 and Example 2A are repeated, except using the formulation 1C as described in Example 1. A similar unexpected improvement in FPF, and its associated advantage, is achieved.

What is claimed is:

1. A pressurized metered dose inhaler (pMDI) comprising:
a. a container;
b. a pharmaceutical formulation under pressure in the container, said pharmaceutical formulation comprising: (i) about from about 75% by weight to about 95% by weight of HFO-1234ze(E); (ii) from about 5% by weight to about 25% by weight of ethanol; and (iii) ipratropium bromide in solution in said ethanol and/or said HFO-1234ze; and
c. a normally closed valve on the container
d. an actuator fluidly connected to the valve and having an orifice diameter of less than 0.5 mm, said valve being actuatable to an open position to release said pharmaceutical formulation to pass through said orifice and produce an aerosol spray containing ipratropium bromide in solution.

2. The pMDI of claim 1 wherein said pharmaceutical composition further comprises from greater than 0.005% by weight to less than 0.015% by weight of a stabilizer and from 0.1% by weight to about 1.5% by weight of water.

3. The pMDI of claim 2 wherein said stabilizer comprises citric acid.

4. The pMDI of claim 1 wherein said actuator orifice has a diameter of from greater than 0.1 mm to less than 0.45 mm.

5. The pMDI of claim 1 wherein said actuator orifice has a diameter of from greater than 0.15 mm to less than 0.4 mm.

6. The pMDI of claim 1 wherein said actuator orifice has a diameter of from greater than 0.15 mm to less than 0.35 mm.

7. The pMDI of claim 2 wherein said pharmaceutical formulation comprises: (i) about from about 80% by weight to about 87% by weight of HFO-1234ze(E); (ii) from about 12% by weight to about 20% by weight of ethanol; and (iii) ipratropium bromide in solution in said ethanol and/or said HFO-1234ze and/or said water.

8. The pMDI of claim 2 wherein said pharmaceutical formulation comprises: (i) about from about 85% by weight to about 95% by weight of HFO-1234ze(E); (ii) from about 5% by weight to about 15% by weight of ethanol; and (iii) ipratropium bromide in solution in said ethanol and/or said HFO-1234ze and/or said water.

9. The pMDI of claim 8 wherein said actuator orifice has a diameter of from greater than 0.15 mm to less than 0.35 mm.

10. A method of delivering a dose of ipratropium bromide to the respiratory tract of an animal comprising:
a. providing in a pMDI comprising:
i. a canister;
ii. a formulation comprising ipratropium bromide in solution contained in the canister, said formulation comprising: (1) from about 75% by weight to about 88% by weight of HFO-1234ze(E); (2) from about 13% by weight to about 25% by weight of ethanol; and (3) ipratropium bromide; and
iii. a metering valve releasably closing the canister and having orifice diameter of less than 0.5 mm; and
b. actuating said pMDI to produce an aerosol spray of said ipratropium bromide.

11. The method of claim 10 wherein said actuating of said pMDI comprises actuating said pMDI to produce said aerosol spray and wherein said aerosol spray has a fine particle fraction of greater than about 40%.

12. The method of claim 10 wherein said actuating of said pMDI comprises actuating said pMDI to produce said aerosol spray and wherein said aerosol spray has a fine particle fraction of greater than about 45%.

13. The method of claim 12 wherein said actuator orifice has a diameter of from greater than 0.15 mm to less than 0.35 mm.

14. The method of claim 10 wherein said pharmaceutical formulation comprises: (i) about from about 80% by weight to about 87% by weight of HFO-1234ze(E); (ii) from about 13% by weight to about 18% by weight of ethanol; and (iii) ipratropium bromide in solution in said ethanol and/or said HFO-1234ze.

15. The method of claim 10 wherein said pharmaceutical formulation comprises: (i) about from about 82% by weight to about 87% by weight of HFO-1234ze(E); (ii) from about 13% by weight to about 18% by weight of ethanol; and (iii) ipratropium bromide in solution in said ethanol and/or said HFO-1234ze.

16. The method of claim 10 wherein said pharmaceutical formulation comprises: (i) about from about 83% by weight to about 86% by weight of HFO-1234ze(E); (ii) from about 14% by weight to about 17% by weight of ethanol; and (iii) ipratropium bromide in solution in said ethanol and/or said HFO-1234ze.

17. The method of claim 10 wherein said pharmaceutical formulation comprises: (i) about from about 84% by weight to about 86% by weight of HFO-1234ze(E); (ii) from about 14% by weight to about 16% by weight of ethanol; and (iii) ipratropium bromide in solution in said ethanol and/or said HFO-1234ze.

18. The method of claim 10 wherein said pharmaceutical formulation comprises: (i) about 84% by weight of HFO-1234ze(E); (ii) about 16% by weight of ethanol; and (iii) ipratropium bromide in solution in said ethanol and/or said HFO-1234ze.

19. A pharmaceutical composition in the form of an aerosol spray comprising particles comprising ipratropium bromide and wherein greater than about 40% by weight of said spray of particles are particles having a size of 5 microns or less.

20. The pharmaceutical composition of claim 9 wherein greater than about 45% by weight of said spray of particles are particles having a size of 5 microns or less.
